(19) United States
(12) Patent Application Publication
(10) Pub. No.: US 2013/0030410 A1
(43) Pub. Date: Jan. 31, 2013

(54) Venous heated ablation catheter

(71) Applicants: William Joseph Drasler, Minnetonka, MN (US); Kevin Leroy Nickels, Wayzata, MN (US); Edward James Black, Woodbury, MN (US)

(72) Inventors: William Joseph Drasler, Minnetonka, MN (US); Kevin Leroy Nickels, Wayzata, MN (US); Edward James Black, Woodbury, MN (US)

(21) Appl. No.: 13/373,634

(22) Filed: Nov. 22, 2011

Related U.S. Application Data
(60) Provisional application No. 61/458,394, filed on Nov. 23, 2010.
(60) Provisional application No. 61/575,530, filed on Aug. 23, 2011.

(51) Int. Cl. A61M 25/10

(52) U.S. Cl. 604/510; 604/96.01; 604/101.01

(57) ABSTRACT

A catheter for delivery of sclerosant to a tubular member or vein of the body to cause ablation. The catheter has one or more balloons located near the distal end to enhance effectiveness of the sclerosant and prevent its delivery to the deep venous system. The catheter has an orifice located near the distal end of the catheter to direct the sclerosant into the vessel and one or more effluent openings to provide for removal of the sclerosant fluid. A heating member such as an electrical resistance element, a Laser probe, or an RF electrode located within the catheter lumen or on the outside of the catheter shaft heats the sclerosant fluid to improve its ablation effectiveness.

### CROSS REFERENCE TO RELATED APPLICATIONS

This nonprovisional patent application makes reference to and includes information found in the provisional patent applications No. 61/458,394 entitled “Venous Ablation Catheter” filed 23 Nov. 2010 by William J. Drasler, “Venous Perforator Ablation Catheter” filed 22 Aug. 2011 by William J. Drasler, and No. 61/575,530 entitled “Venous Heated Ablation Catheter” filed 23 Aug. 2011 by William J. Drasler, Kevin Nickels, and Edward Black.

### FIELD OF THE INVENTION

This invention relates to an interventional device that is placed into a lumen of the body such as a vein to ablate or occlude the venous lumen.

### BACKGROUND OF THE INVENTION

Superficial veins either below or above the knee can develop incompetent valves with an inability to direct blood from the superficial venous system into the deep system and result in reflux of blood from the deep system into the superficial system. Such reflux can occur at the junction of a superficial vein such as the saphenous vein with the deep veins such as the femoral vein or the popliteal vein. Also reflux can occur between the superficial venous system and the deep system through incompetent valves associated with perforator veins. This reflux of blood can result in varicose veins and superficial venous insufficiency.

One way to treat this problem is by ablating the superficial veins. Several devices and methods have been used to ablate the saphenous vein including endovascular laser therapy (EVLT) and endovascular radiofrequency ablation (RFA). Such devices are expensive and are not often effective in maintaining long term occlusion of larger saphenous veins. Also, both EVLT and RFA therapies can generate enough heat to cause pain or neuropathy in nearby nerves or skin trauma in the lower leg and thereby these therapies require the use of tumescent anesthesia to overcome these negative clinical sequellae. Another way of occluding such veins is by use of sclerotherapy. With sclerotherapy a liquid sclerosant or a foam sclerosant is injected into the vein to cause trauma to the endothelial lining of the vessel resulting in occlusion of the vein. Such systems are difficult to control and can result in the sclerosant flowing into the deep venous system potentially causing trauma to this important deep system which is necessary for venous return of blood from the leg to the heart. The foam sclerosant can also be inconsistent in the size of the bubbles that make up the foam. It is also not easy for the operator to control the amount of sclerosant that is delivered to the vein and to ensure that the sclerosant does not migrate through the patient's venous system back to his heart, lungs, and potentially back to his brain or other important vessels of the body.

Another way of treating reflux from the deep venous system to the superficial system is to ablate or ligate the perforator vein. Access to the perforator vein can be obtained directly through the skin and through the subcutaneous tissues. This method has been performed by both EVLT and RFA technologies but has not been successful at providing a durable solution. Another approach for treating the perforator veins is via subfascial endoscopic perforator surgery (SEPS). This procedure is complex and is not performed often due to patient discomfort and its high expense. What is needed is a device and method that can ablate a perforator vein in a low cost manner that is durable over a long period of time.

### SUMMARY

The present invention is well suited to use as an interventional catheter for occlusive treatment of the superficial vessels of the leg. The invention can also be applied to other tubular members of the body including arteries, veins, ducts, air passages, and other fluid ducts of the body. For tubular ducts of the body the present invention can be used to deliver a medium such as a liquid, fluid, solution or suspension in a controlled manner to a specific region of the tubular member with the potential for removal of such medium from the vessel if desired. The medium can be, for example, a chemotherapeutic agent, contrast agent, diagnostic agent, ablative agent or any other medium that is required for delivery in a controlled manner to a specific region of the tubular member of the body.

One application for the present invention is in the veins located in the leg. The catheter enters the venous system at either a proximal location and is advanced distally in the leg or can be entered in a distal venous location and advance proximally. The catheter delivers a sclerosant mixture or sclerosant solution to the lumen of the vein. The sclerosing solution exits the catheter through an orifice as a sclerosant solution or foam, and is brought into contact with the venous wall where it is recirculated between two balloons that partially contain the sclerosant. The sclerosant is then evacuated from an evacuation port such that the amount of sclerosant that is delivered to the lumen is approximately removed from the lumen. Additional sclerosant can be delivered to the lumen to form a net positive inflow, or alternately additional sclerosant can be removed from the venous lumen to generate a net volume outflow from the vein. The catheter can be withdrawn slowly in the venous lumen thereby providing the venous wall with exposure to the sclerosant agent; the sclerosant agent is delivered in a controlled manner that will not result in migration to the deep venous system; and the sclerosant will be evacuated thereby removing negative sequellae associated with unwanted migration of the sclerosant to the rest of the body.

In one embodiment a solution containing the sclerosant mixed with saline and dissolved carbon dioxide (CO2) is delivered via a small tubing or hypo tube contained within the catheter shaft to a region located between two balloon located at the distal end of the catheter. The distal balloon can be somewhat smaller in diameter than the proximal balloon. Upon passage through a side orifice the CO2 is removed from solution and the highly energetic passage of the fluid jet through the side orifice causes the sclerosant to form a foam that contains the CO2 gas in the form of well-formed micro bubbles of a consistent size. The microfoam sclerosant is then driven into a recirculation pattern caused by the side fluid jet which comes into direct contact with the venous wall to ablate the venous wall tissue.

An additional fluid jet located at the distal end of the catheter directs a second stream of fluid back in a proximal direction at the open distal end of the catheter; the high velocity distal jet creates a local vacuum that draws surrounding fluid into the jet, including the microfoam that was recirculating between the two balloons, into the low pressure region of the jet. The jet also creates a stagnation pressure to push the microfoam out of the catheter shaft and out of the body. The distal fluid jet can be formed from the same solution as the side jet that is used to form the foam that comes out of the side orifice if desired. Alternately, the distal jet can be a clean-up jet that allows a saline wash of the venous lumen if desired.

A control pump controls the amount of fluid effluent that is evacuated from the catheter via the catheter shaft. Adjusting the amount of effluent versus the amount of inflow from the side jet and the distal jet can be used to control the net fluid input into the vein or output from the vein. The net fluid delivery to the vein can be a positive input or it can be a net outflow from the vein or it can be a zero net fluid delivery.

In another embodiment a liquid sclerosant or a solution of sclerosant and saline without CO2 is expelled out of the side orifice between the two balloons. The sclerosant solution can be delivered via a small tube that is contained in the catheter shaft. The sclerosant solution is then recirculated into contact with the venous wall and then drawn toward the distal end of the catheter where it is removed via the distal jet as described in the first embodiment.

In still another embodiment, the sclerosant solution either with or without the CO2 can be delivered by from the catheter from between two balloons, but then move proximally to an evacuation port where the sclerosant is removed. An additional proximal jet can be placed at this proximal port to generate a stagnation pressure and assist in removal of the sclerosant, rather than having the distal jet.

In yet other embodiments, the catheter of the present invention can be formed using only one balloon instead of two balloons at the distal end of the catheter or with three or more balloons instead of two. Furthermore, the distal or proximal jet that is used to generate stagnation pressure could be replaced by a vacuum system or vacuum pump that is attached to the central lumen of the catheter shaft and used for removal of the sclerosant. Yet furthermore, the sclerosant that is removed from the catheter can be returned back to the manifold of the catheter and pumped back to the side, distal, or proximal orifice and back into the vessel lumen for a second or third contact with the venous wall in order to reuse the sclerosant solution or sclerosing agent if desired.

Typical sclerosing agents currently used in the clinic include sodium tetradecyl sulfate (STS), polidocanol (POL) and others. Liquid sclerosing agents are typically used clinically at a concentration that ranges from 0.5-4% depending upon the sclerosing agent and the size and type of vessel being treated. Lower concentrations of sclerosing agents ranging from 0.1-0.5% are also used for sclerosing smaller veins such as spider veins. When forming a foam using conventional techniques, one part of the liquid sclerosing solution is mixed with four parts of sterile room air or other gas. In the present invention CO2 gas is dissolved in the sclerosing solution and supplied to the present catheter as a solution.

In a yet further embodiment, hot saline, hot sterile water, hot fluid, heated solution, steam, or a heated sclerosant solution can be used for delivery out of the side or distal orifice of the present catheter. Contact of the hot saline, fluid, or solution with the vessel wall will cause vessel necrosis and lead to scarring of the venous wall leading to an occluded vein. The hot saline would be evacuated in a manner described earlier for removal of the sclerosant.

In combination with the sclerosant treatment of the venous conduit it may be desirable to place an occluding member into the vein. Placement of an occluding member near the saphenofemoral junction (SFJ) or saphenopopliteal junction (SPJ) can help to reduce the possibility of thrombus migration from the superficial vein into the deep venous system. The requirement of such an occlusion element is that it be easy to apply percutaneously and would not be large and bulky since there is significant bending motion near the SFJ and SPJ.

One embodiment for such a device is a balloon that is formed out of a biodegradable material such as a pericardial tissue or collagen tissue. The flat sheet of pericardium can be formed via increased temperature and pressure with a mold to form it into a balloon shape or a half of a balloon. The two half balloon portions can be attached together via a biodegradable glue or suture to form a complete balloon. Alternately, a slurry or solution of collagen or biodegradable materials can be cast into a mold that forms a balloon or a portion or portions of a balloon that can be later attached together to form a balloon. The biodegradable balloon would be filled with a saline solution and would be expanded into the lumen of the saphenous vein near the SFJ. The saline solution can contain ions or molecules that assist in reducing the potential for infection. Tissue from the venous wall surrounding the balloon would be allowed to ingrow into the balloon wall thereby fixing or attaching the balloon wall to the venous wall. After a few weeks, the balloon wall would be infiltrated by cellular ingrowth and the saline would gradually be removed or released. The net result is an occluded vein with a soft pliable biodegradable balloon located in its lumen, and attached to the venous wall.

In further one embodiment the catheter of the present invention can be delivered across the skin and through the subcutaneous tissues directly into the lumen of the perforator vein or into another vein. This access location into the vein can be at the junction of the perforator vein with the superficial vein or it can be deeper to the tissue of the treated limb and into the lumen of the perforator vein. The catheter can have a very low profile of approximately 1 mm diameter. A distal balloon can be inflated to ensure that sclerosant solution or fluid does not enter into the deep venous system. The distal balloon can be inflated in the deep vein or in the perforator vein near the deep vein. A side jet of sclerosant can be directed outward against the vessel wall to enhance vessel trauma and enhance the effectiveness of the sclerosant solution. The sclerosant solution or fluid can be heated, for example, to approximately 47 degrees Celsius or more, to further enhance the effectiveness of the sclerosant on the venous wall necrosis and fibrosis. This catheter can alternately be advanced through the deep venous system to gain access into the perforator vein.

In an additional further embodiment of the catheter, a separate guidewire lumen can be provided through the catheter shaft to assist in providing access to the perforator vein. The catheter can therein follow over a guidewire that is placed through the skin and further advanced through the perforator vein; alternately the guidewire can extend into the perforator vein from the deep venous system and the catheter can follow over the wire from the deep system into the perforator vein. An effluent opening can be located along the shaft of the catheter to provide an opening for removal of sclerosant fluid from the perforator.

In addition to the distal balloon, a proximal balloon can be located on the catheter shaft just proximal to the effluent opening. This proximal balloon can be similar in diameter to the distal balloon. In this way the sclerosant fluid is contained within the recirculation region and recirculated between the distal and proximal balloons to expose the perforator vein wall to a controlled dose of sclerosant exposure for more consistent vein ablation. This recirculating sclerosant can be heated to improve the effectiveness of the sclerosing agent. Either the distal balloon, the proximal balloon, or both balloons can have a roughed surface that is intended to come into contact with the vein wall; relative movement between the roughened surface and the vein wall will cause abrasion of the vein wall and will render the vein wall more susceptible to necrosis and fibroses from exposure to the sclerosing fluid.

In yet an additional further embodiment for the catheter, a jet can direct a radial stream of sclerosant fluid outward from one or more side orifices against the vein wall between a distal and proximal balloon. In one embodiment the proximal balloon is smaller in diameter than the distal balloon. An effluent opening located just proximal to the proximal balloon provides a site for the removal of the sclerosant effluent. The smaller diameter proximal balloon helps to place the sclerosant fluid into intimate contact with the vein wall along its entire perimeter.

Several types of pumps can be used to provide the sclerosant to the inlet port of the catheter for delivery to the side orifices for outward radial spray onto the vein walls. A positive displacement pump such as a piston pump or a roller pump can be used for this purpose. Alternately a centrifugal pump could also be used. Similarly, the same types of pumps can be used to provide effluent flow or control the flow rate for effluent removal.

Heating methods have been contemplated for heating the sclerosant fluid prior to delivery by the supply tube to the side orifice and into contact with the vein wall. The heating member can be an electrical resistance heating coil or a high resistance heating wire placed into contact with the sclerosing fluid as it is being delivered from the supply pump through the supply tube to the delivery catheter. Alternately, the heating member can be placed within the catheter shaft at a location near its distal end to heat the sclerosing fluid. As another option, the heating member can be located on the outside of the catheter shaft near the distal end such that it heats the fluid within the perforator vein directly. The heated fluid can include blood tissue along with sclerosing fluid.

Placement of a heating member in the supply tube that delivers sclerosing fluid to the vessel wall will enhance the effectiveness of the sclerosant. The temperature necessary to provide improved effectiveness is approximately 5-25 degrees Celsius above body temperature; higher temperatures can also be used. At higher temperatures, fluid handling can become more difficult and the pain and neuropathy associated with higher temperature vein ablation would require tumescent anesthesia to be administered.

In yet another embodiment for the catheter of the present invention, a very low profile delivery catheter is presented that follows over a guidewire that extends from a superficial vein and into a perforator vein and further into the deep venous system. The catheter has two balloons positioned near its distal end and less than 3 cm apart. A distal balloon prevents sclerosant fluid from entering into the deep venous system and can make a seal around the guidewire or at the distal end of the catheter such that the guidewire lumen can be used for removal of effluent sclerosant. The optional proximal balloon can be used to prevent sclerosant fluid from entering the superficial system for those patients that have a functional superficial venous system and a dysfunctional perforator vein. This catheter can also be delivered to the perforator vein or to the superficial vein from the deep venous system if desired.

In still another embodiment, the guidewire that is used to extend from the superficial vein into the perforator vein and into the deep venous system provides the distal occlusion to prevent sclerosant from entering the deep venous system. A balloon located on the distal portion of hollow guidewire body provides passage for the inflation fluid. A very low profile catheter with a proximal occlusive balloon can then follow over the guidewire (OTW) body through the superficial vein and into the perforator vein. The low profile of the distal occlusive guidewire and the low profile proximal occlusive OTW catheter allow entry into a superficial vein at a remote site located several centimeters away from the location of the incompetent perforator vein which often can be associated with a venous ulcer. Treatment of the perforator vein from a remote site is safer for the patient with reduced likelihood of infection transmission within the body.

In yet another embodiment of the present invention the catheter can be used to deliver steam as a form of sclerosant solution or sclerosant fluid or sclerosant. The RF, Laser, or Electric resistance heating energy supply described in the embodiments of the present invention can be used to heat water and generate steam which is then delivered into contact with the internal lumen of the vein to be treated. The steam then condenses within the vein lumen thereby delivering its latent heat of vaporization to the vessel wall and causing trauma to the endothelium and underlying layers of the vein wall. The steam can be created at the proximal or distal end of the catheter and delivered to the distal end of the catheter. In one preferred embodiment, the steam is created by a heating member located near the distal end of the catheter on the inside or the outside of the catheter. Any of the embodiments for delivery catheter of the present invention can comprise a heating element and one of the embodied energy sources to provide a sclerosing fluid that includes steam.

In another embodiment for the heating member radiofrequency (RF) heating can be delivered to the sclerosing fluid traveling in the supply tube that connects the supply pump with the delivery catheter. The RF heating element can be a pair of metal electrodes placed within the supply tube to generate an oscillating electromagnetic energy that is transferred to polar molecules such as water contained within the sclerosing fluid. Alternately, a coil can be placed into the supply tube to couple to the polar molecules contained in the sclerosing fluid via the electromagnetic field produced by the coil. The RF heating member can alternately be located within the catheter shaft or around the outside of the catheter shaft near the distal end of the delivery catheter. The RF heating member can be a bipolar electrode or it can be a unipolar electrode with a counter electrode placed a distance away on the skin surface or within the limb being treated.

An additional embodiment for the heating member includes a laser probe that receives energy from a laser energy supply such as a diode laser. The laser probe can be placed into contact with the sclerosing fluid found in the supply tube. An energy transmission conduit such as fiber optic cable can connect the heating member with the laser energy supply. A diode laser or other laser operating at approximately 1320-1470 nm wavelength will produce energy that is absorbed readily by water molecules found within the sclerosing fluid such that the fluid will heat rapidly. A laser heating member can alternately be located at a distal location of the delivery catheter either within the catheter shaft or on the outside of the catheter shaft in direct contact with the fluid contained within the perforator vein. A laser located in this location could also operate with a wavelength ranging from approximately 810-980 nm to absorb more efficiently in hemoglobin molecules. The heating of the sclerosing fluid by either RF heating member or via a laser heating member will cause the sclerosing fluid or the heated blood tissue to be more effective in ablating the wall of the vein and result in a more durable therapy.

Other forms of heat generation have been contemplated and can be used to provide thermal energy to a heating member for heating a fluid such as a sclerosant fluid to a temperature that is more effective. For example, chemical mixtures have been known to produce exothermic reactions that can be used to raise the temperature of the sclerosing fluid, other medium, or the chemical mixture itself that is delivered to a tubular member. It is understood that other heating means known in the industry can be used to heat the sclerosing fluid or other medium to a higher temperature.

In one or more embodiments an RF, laser, electrical resistance, or other heating member is used to heat water molecules to generate steam which then becomes the sclerosing fluid that is used to ablate the vein wall. The steam can be isolated between two balloons to prevent the steam from reaching the deep venous system or other veins that are not intended to be ablated.

### BRIEF DESCRIPTION OF THE DRAWINGS

FIG. 1A is a plan view of the delivery catheter with one larger sealing balloon and one smaller diameter balloon and an orifice located between the balloons.

FIG. 1B is a plan view of the sclerosant fluid delivery and control system.

FIG. 2 is a plan view of the delivery catheter with a distal sealing balloon and being operated within a vein.

FIG. 3 is a plan view of an implantable occlusion balloon mounted on a catheter shaft for delivery to a vein.

FIG. 4 is a plan view of the implantable occlusion balloon showing its proximal and distal seal with a guidewire during delivery to a vein.

FIG. 5 is a plan view of a deflated occlusion balloon being delivered to a vein lumen.

FIG. 5B is a plan view of an inflated occlusion balloon delivered to a vein lumen.

FIG. 6A is a plan view of a delivery catheter having a distal balloon located within the catheter shaft and having an ultrasound marker.

FIG. 6B is a plan view of the distal portion of a delivery catheter with the distal balloon inflated.

FIG. 7A is a plan view of a delivery catheter having a separate guidewire lumen and a distal balloon in a noninflated configuration.

FIG. 7B is a plan view of the distal portion of a delivery catheter with the distal balloon located near the distal end of the catheter shaft in an inflated condition.

FIG. 7C is a plan view of the distal portion of a delivery catheter having a proximal balloon located near the distal end of the catheter shaft.

FIG. 8A is a plan view of the delivery catheter having a distal balloon located within the catheter shaft in a noninflated configuration.

FIG. 8B is a plan view of the distal portion of a delivery catheter with the distal balloon inflated.

FIG. 9A is a plan view of the delivery catheter having both a proximal and distal balloon in a deflated condition located near the distal end of the catheter and having a side orifice and effluent opening.

FIG. 9B is a plan view of the delivery catheter having both a proximal and distal balloon in an inflated condition near the distal end of the catheter and having a side orifice and effluent opening.

FIG. 9C is a plan view of the distal portion of the delivery catheter having two balloons located near the distal end and having a roughened surface and being moved in an axial direction.

FIG. 9D is a plan view of the distal portion of the delivery catheter having two balloons located near the distal end and having a roughened surface and being moved in an rotational direction.

FIG. 10A is a delivery catheter having multiple side orifices for sclerosant fluid delivery and having two balloons which are deflated with the distal balloon located within the catheter shaft.

FIG. 10B is a delivery catheter having multiple side orifices for sclerosant fluid delivery and having two balloons which are inflated and not the same diameter in the venous system of the body.

FIG. 11A is a plan view of a delivery catheter having a distal balloon positioned within the deep vein or the distal portion of a perforator vein.

FIG. 11B is a plan view of a delivery catheter having two similarly sized sealing balloons positioned within the perforator vein and an orifice and effluent opening located between the balloons.

FIG. 11C is a plan view of a delivery catheter having two different sized balloons positioned within the perforator vein and an effluent opening located proximal to the proximal balloon.

FIG. 11D is a plan view of a delivery catheter being introduced into the deep venous system and extending into the perforator vein with a proximal balloon forming a seal to ensure sclerosant does not enter the deep vein.

FIG. 12A is a plan view of sclerosant delivery system to a delivery catheter where the control pump and supply pump are both the same piston pump to ensure a balance of sclerosant inflow and outflow, and a heating member is located within the supply tube which is in fluid communication with the sclerosant fluid within the catheter shaft.

FIG. 12B is a plan view of delivery catheter and a supply pump and a control pump including a pressure transducer to indicate that sclerosant fluid is being removed from the vein.

FIG. 13A is a plan view of a distal portion of a delivery catheter having a heating member located near the distal end of the catheter shaft and located on the outside of the catheter shaft.

FIG. 13B is a plan view of a distal portion of a delivery catheter having a heating member located near the distal end of the catheter shaft and located within the catheter shaft.

FIG. 14A is a plan view of delivery catheter, the supply system, the control system, and an energy supply that supplies energy to the heating member located in the supply tube which is in fluid communication with the sclerosant fluid within the fluid sclerosant lumen of the catheter shaft.

FIG. 14B is a plan view of the delivery catheter having the heating member located near the distal end of the catheter shaft.

FIG. 15B is a plan view of a delivery catheter having a distal balloon located at an open distal end of the catheter shaft to form a seal with the vein wall and with the guidewire.

FIG. 15B is a plan view of a delivery catheter having a proximal and distal balloon and an open distal end for passage of a guidewire.

FIG. 15C is a plan view of a guidewire passing directly through the skin and into a perforator vein to provide a member to travel over for a delivery catheter.

FIG. 15D is a plan view of a guidewire passing through the skin and following along a superficial vein and turning into a perforator vein to provide a member to travel over for a delivery catheter.

FIG. 15E is a plan view of a delivery catheter travelling over a guidewire that is positioned within a perforator vein to a site within the perforator vein that forms a recirculation region that is isolated from the deep venous system.

FIG. 16 is a plan view of a distal occlusive guidewire that has a deflated distal guidewire balloon that can be used to protect the deep vein from sclerosant fluid when inflated.

FIG. 17 is a plan view of a delivery catheter that is intended to pass over a guidewire such as the one in FIG. 16 and deliver a sclerosant fluid and remove the sclerosant fluid through its open distal end.

FIG. 18 is a plan view of the delivery catheter of FIG. 17 travelling over the distal occlusive guidewire of FIG. 16 where the distal guidewire balloon is located in the deep vein and the delivery catheter is ablating a perforator vein.

FIG. 19A is a plan view of the supply tube showing a radiofrequency electrode heating member located within the supply tube and a transmission conduit connecting the heating member with the electrical energy supply.

FIG. 19B is a plan view of the supply tube showing a radiofrequency coil heating member located within the supply tube and a transmission conduit connecting the RF heating member with the RF energy supply.

FIG. 19C is a plan view of the supply tube showing a laser probe heating member located within the supply tube and a transmission conduit such as an optical fiber connecting the heating member with the laser energy supply.

FIG. 20A is a plan view of the distal portion of a delivery catheter having a radiofrequency electrode heating member located within the sclerosant fluid lumen of the catheter shaft.

FIG. 20B is a plan view of the distal portion of a delivery catheter having a radiofrequency electrode heating member located around the outside of the catheter shaft and in fluid communication with the sclerosant fluid lumen located within the catheter shaft.

FIG. 20C is a plan view of the distal portion of a delivery catheter having a radiofrequency coil heating member located within the sclerosant fluid lumen of the catheter shaft.

FIG. 20D is a plan view of the distal portion of a delivery catheter having a radiofrequency coil heating member located around the outside of the catheter shaft and in fluid communication with the sclerosant fluid lumen located within the catheter shaft.

FIG. 20E is a plan view of the distal portion of a delivery catheter having a laser probe heating member located within the sclerosant fluid lumen of the catheter shaft.

FIG. 20F is a plan view of the distal portion of a delivery catheter having a laser probe heating member located around the outside of the catheter shaft and in fluid communication with the sclerosant fluid lumen located within the catheter shaft.

### DETAILED DESCRIPTION OF THE INVENTION

The present invention is an interventional catheter for delivery of the sclerosant solution to the venous system of the body. The catheter provides a controlled delivery of a sclerosant solution and removal of the sclerosant such that the superficial vein and nearby perforating veins are occluded and the sclerosant has not migrated into the deep venous system or to other parts of the body.

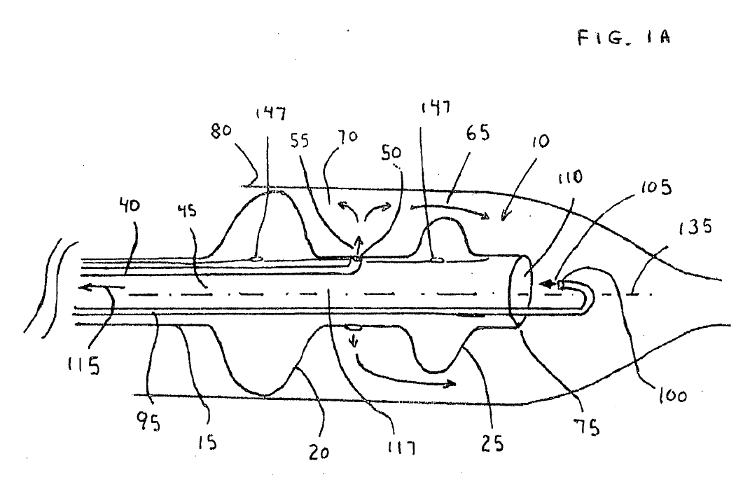

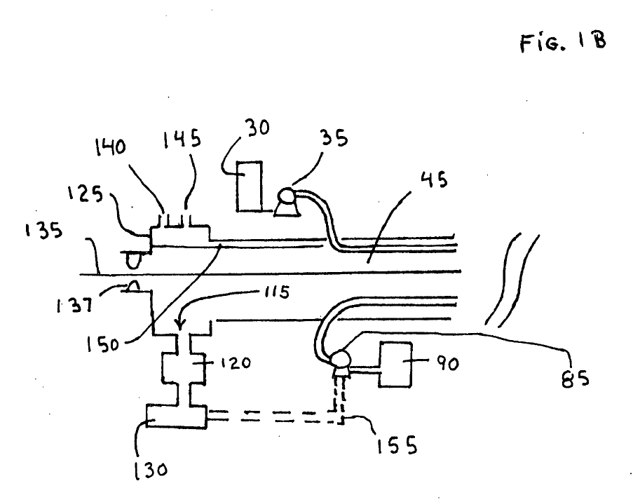

FIGS. 1A and 1B show a first embodiment of the delivery catheter (10). The delivery catheter (10) has a catheter shaft (15) with two balloons located at its distal end (75), a proximal balloon (20) and a distal balloon (25), the distal balloon (25) being somewhat smaller in diameter than the proximal balloon (20). The proximal balloon (20) makes contact with the vein to be treated and forms a seal along the perimeter of the balloon. A sclerosant solution is delivered from a first reservoir (30) by a first pump (35) to a side tube (40) located in the shaft lumen (45) to a side orifice (50) located between the balloons and directed outwards to form a side jet (55). The sclerosant solution can be formed from a sclerosing agent such as STS, POL, sodium morrhuate, ethanolamine oleate, glycerin, hypertonic saline, mixtures with alcohol or dextrose, iodine compounds, or other known sclerosing agents mixed with saline and having a CO2 gas or other soluble gas dissolved in the solution. Upon the side jet (55) exiting the side orifice (50), the CO2 comes out of solution and mixes with the sclerosing agent to form well defined bubbles of sclerosant foam with CO2 inside of them. The bubble size is consistent in size and is formed at the time that it is injected directly into the vein lumen (60) of the vein being ablated. Typically the bubble size for a microfoam is smaller than 100 microns and for the microbubbles formed by the waterjet, the size is consistently smaller than 100 microns. Alternately, the sclerosing solution can be a sclerosing agent or a sclerosing agent mixed with saline, but without CO2. The exiting side jet (55) forms a recirculation pattern (65) in a recirculation region (70) located between the two balloons and then recirculating sclerosing solution escapes the recirculation pattern (65) to move distally past the distal balloon (25) toward the distal end (75) of the catheter. The two balloons help to hold the vein wall (80) away from the outward shooting side jet (55) and control the recirculating sclerosant solution or foam into contact with the vein wall (80) in a controlled volume or space identified by the proximal and distal balloons.

A second pump (85) delivers fluid from a second pump (90) to a distal tube (95) located in the shaft lumen (45) and to the distal orifice (100) and forms a distal jet (105) that is directed at the distal open end (110) of the catheter. This fluid can be the same sclerosant solution that is used to form the side jet (55) or it can be a saline solution or other solution. The high velocity of the distal jet (105) creates a local vacuum that draws the recirculation from the side jet (55) towards the distal jet (105). The distal jet (105) also creates a stagnation pressure at the open distal end (75) of the delivery catheter (10) that serves to push the effluent (115) fluid out of the shaft lumen (45) which also serves as an effluent lumen. A control pump (120) attached to the manifold (125) controls the amount of effluent (115) that is driven from the catheter shaft (15) lumen to the collection reservoir (130). The control pump (120) can control the effluent (115) to be greater than the inflow from the side and distal jet (105) to ensure that the sclerosant solution does not enter into one or more perforator veins or embolize into the deep venous system. The control pump (120) can control the effluent (115) to be less than the inflow from the side jets (55) and distal jets (105) to help push the sclerosant solution into the perforator branches and help to ablate the perforator veins. The net volume input of fluid into the vein being treated can be a net positive or net negative depending upon the goal of the operator, or the fluid flows can be balanced. Such decisions can be made by observing the movement of the foam sclerosing solution under ultrasound guidance.

The delivery catheter (10) can pass over a guidewire (135) to provide passage through the vein being treated; a hemostasis valve (137) located in the manifold (125) ensures that effluent (115) does not escape around the guidewire (135). The delivery catheter (10) can enter the vein at a distal location and can be advanced over the wire to a proximal location, for example, near or distal to the SFJ. The balloons can be inflated using a proximal inflation port (140) and distal inflation port (145) or they can be inflated using a single port that directs inflation fluid via one or more inflation lumens (150) to both of the balloons at the same time. One or more inflation lumens (150) direct balloon inflation medium to one or more balloon openings (147) to inflate the balloons independently or together. The side jet (55) and distal jet (105) can be activated by the first pump (35) and second pump (85) or alternatively, they can be driven using a single pump that delivers fluid to both the side orifice (50) and distal orifice (100). The delivery catheter (10) can then be withdrawn back slowly while the vein wall (80) is being exposed to the sclerosing solution or sclerosing microfoam. Alternately, the delivery catheter (10) can enter at a proximal location in the vein and can be advanced distally; then with the balloons inflated and the side jets (55) and distal jets (105) activated, the delivery catheter (10) can be withdrawn slowly to ablate the vein using the sclerosant solution.

The pressure generated by the first pump (35) and second pump (85) or by a single sclerosant supply pump can range from 500 to 10,000 psi in order to generate a small foam bubble from the side jet (55) exiting the side orifice (50) and to generate a stagnation pressure from the distal jet (105) emanating from the distal orifice (100). The pressure can vary depending upon the viscosity of the fluid and the diameter of the side tube (40) and distal tube (95), and also depends upon the diameter of the side orifice (50) and distal orifice (100). The side orifice (50) for generating a foam and the distal orifice (100) can range in diameter from 0.001 to 0.015 inches. For a side jet (55) that is delivering a fluid that does not form a foam, the diameter of the orifice can be larger if desired and can range from 0.001 to 0.040 inches.

The diameter of the proximal balloon (20) and distal balloon (25) can range in their inflated conformation from approximately 2 cm for a proximal dilated saphenous vein to approximately 3 mm for veins located in the lower leg. A typical balloon size for use above the knee could range from 6-12 mm in diameter. The balloon material should be semicompliant material that would allow the balloon diameter to change by altering the inflation pressure. Such materials include but are not limited to silicone, polyurethane, latex, polyvinylchloride, and others commonly used in the medical device industry.

The first pump (35) and second pump (85) can be positive displacement pumps such as piston pumps, gear pumps, roller pumps, and other pumps generally capable of generating the pressures required to deliver fluid or solution from the first or second pump (90) to the side orifice (50) or distal orifice (100). The control pump (120) can be, for example, a roller pump that is set at a rate that controls the outflow of effluent (115) such that it is in balance with the inflow or alternately, provides a net positive inflow or a net outflow of fluid from the vein. The first pump (35) and second pump (85) can provide a continuous flow to the side and distal orifices or they can provide pulsatile flow to either or both of the side and distal orifices.

The side tube (40) and distal tube (95) can be formed from a metal hypo tube or from a number of plastic members that are capable of supporting the pressure including polyimide tubing. The catheter shaft (15) can be formed from standard plastics used to form catheter tubes used in the industry. The side orifice (50) and distal orifice (100) can be formed by EDM plunge drilling of a hole into a metal hypo tube or it can be formed via standard mechanical drilling of a hole into a plastic tubing.

The delivery of the sclerosant solution in this controlled manner will ablate the entire vein wall (80) around its perimeter but contained partially between the proximal balloon (20) and distal balloon (25). This controlled delivery and exposure of the vessel wall to the sclerosant provides an ability to treat a large diameter saphenous vein. The recirculation pattern (65) will allow the sclerosant to be exposed to the ostium of the perforator branches or extend partway into the perforator vein and cause them also to be effectively ablated without allowing the sclerosing agent to be delivered uncontrollably to the deep venous system. The present invention allows the delivery and removal of sclerosant to be balanced if desired such that the sclerosant solution inflow from the side and distal jets (105) is equal to the outflow of effluent (115) that is controlled by the control pump (120). The control pump (120) can deliver the effluent (115) to a collection reservoir (130). Also, it is possible to cause blood flow from the perforators to move into the superficial system by increasing the exhaust of the effluent (115) as controlled by the control pump (120) in comparison to the inflow from the side jet (55) and distal jet (105). Alternately, the sclerosant can be forced into a portion of the perforator branch by increasing the inflow from the side jets (55) and distal jets (105) such that it is greater than the effluent (115) outflow as controlled by the control pump (120).

It is understood that the inflow can be the sum of the flow from the side tube (40) and the distal tube (95). The same tube can be used to provide flow to both the side tube (40) and the distal tube (95). Also it is understood that even though the device is shown with two balloon, a single balloon could be used, either the proximal or distal balloon (25) without deviation from the present invention.

It is further understood that the effluent (115) can be returned to the catheter as shown in FIG. 1B. If it is desirable to reuse the sclerosant, then the solution can be directed from the control pump (120) over to the second pump (85) via a return tube (155) and then pumped back to the distal orifice (100). It is understood that the effluent (115) could also be returned from the control pump (120) to both the side orifice (50) and the distal orifice (100). It is further understood that the delivery catheter (10) can have more than one side orifices (50) and side jets (55) and can have more than one distal orifices (100) and distal jets (105).

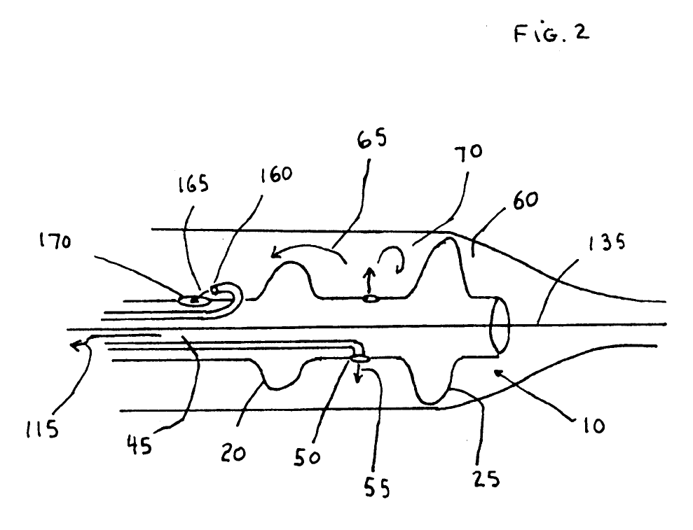

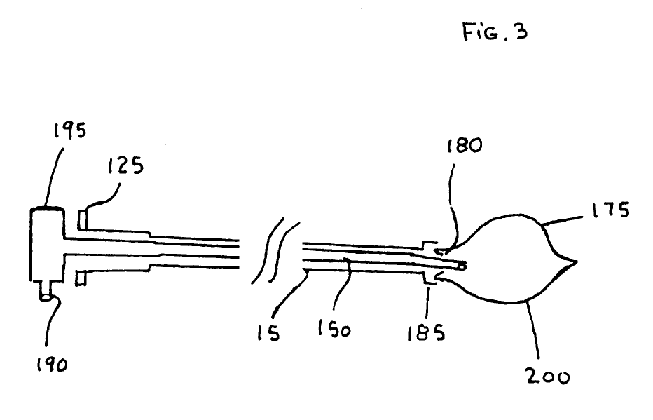

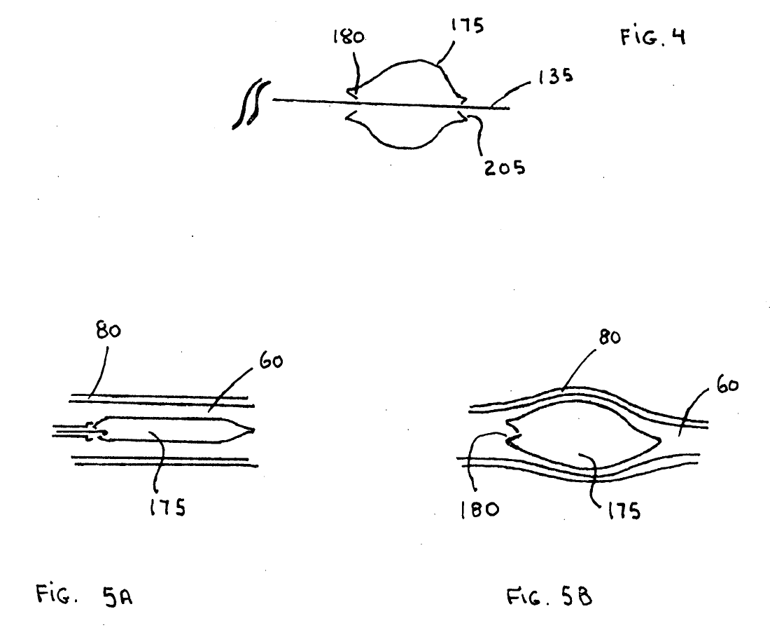

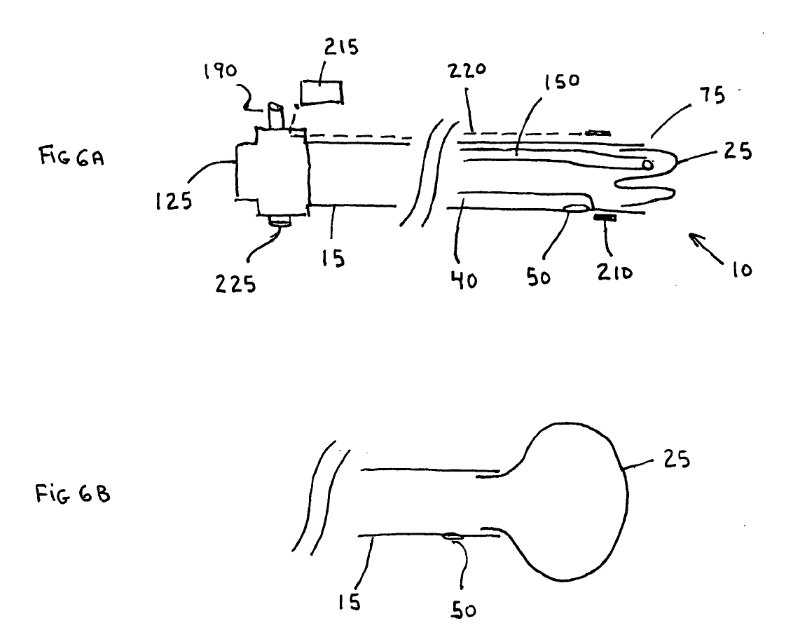

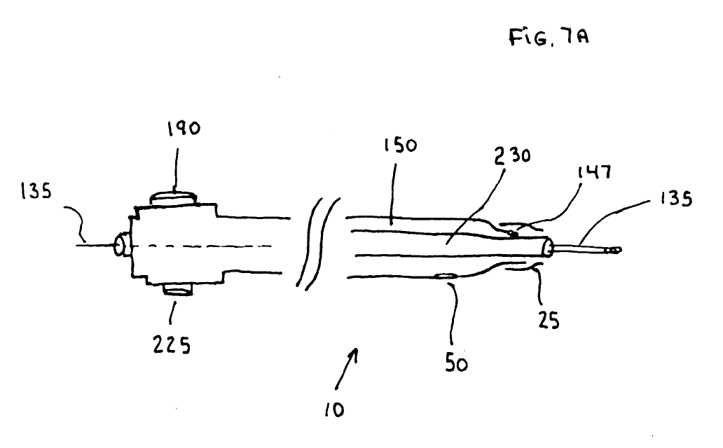

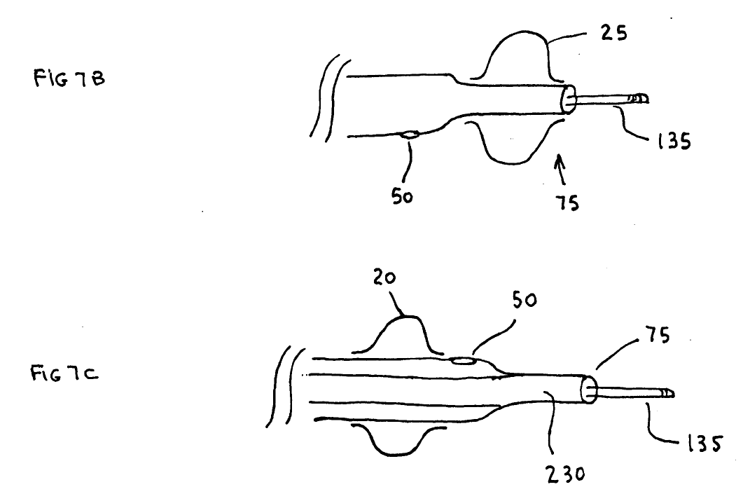

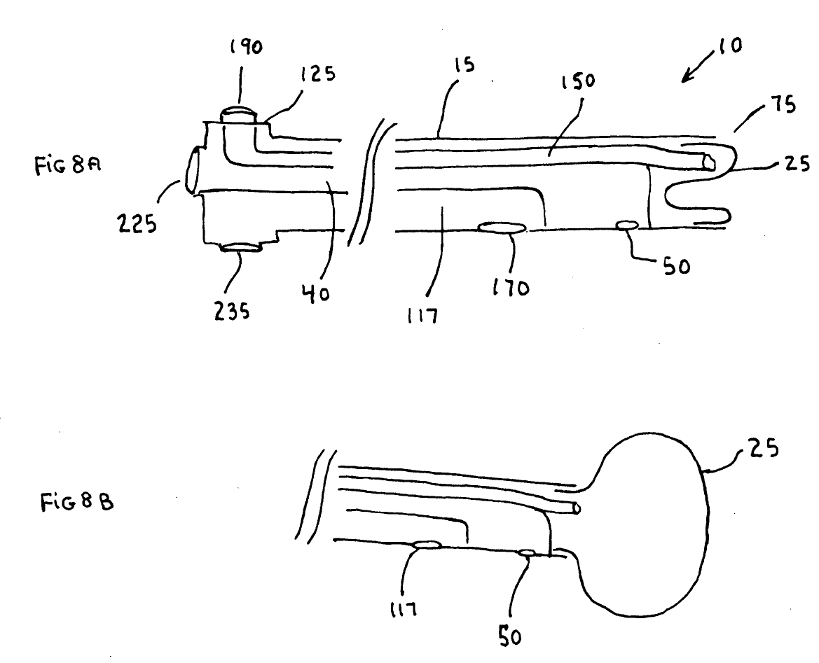

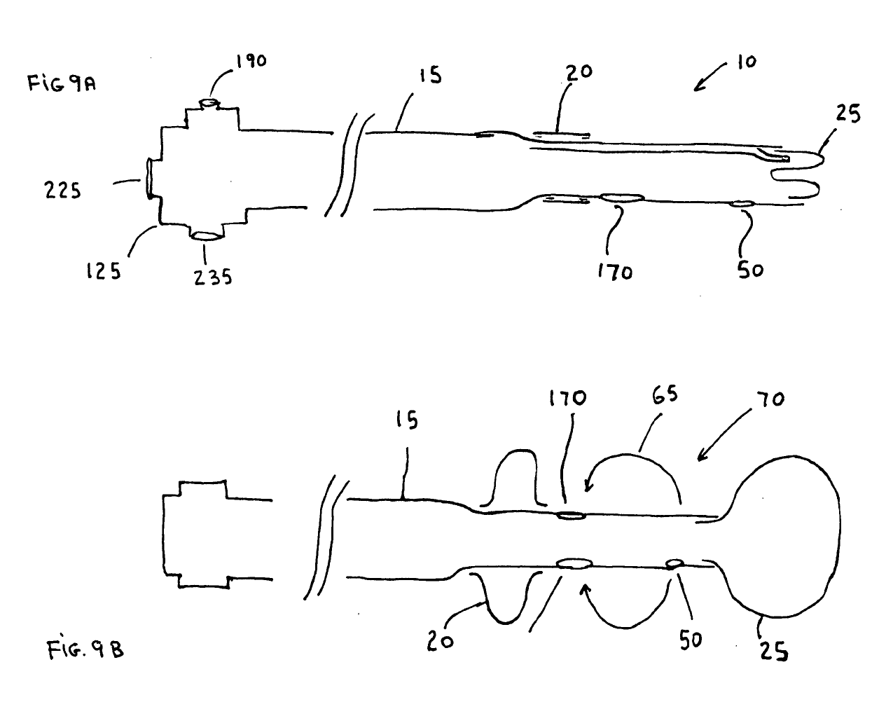

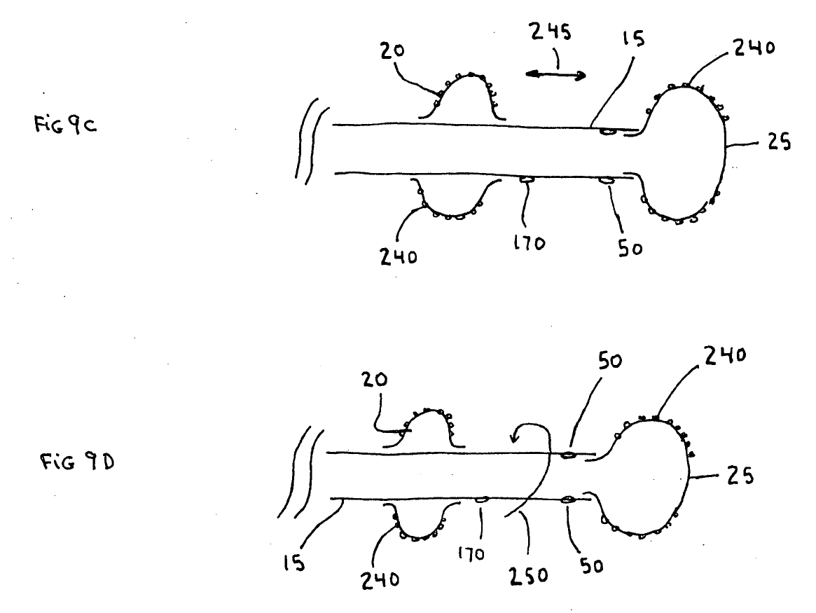

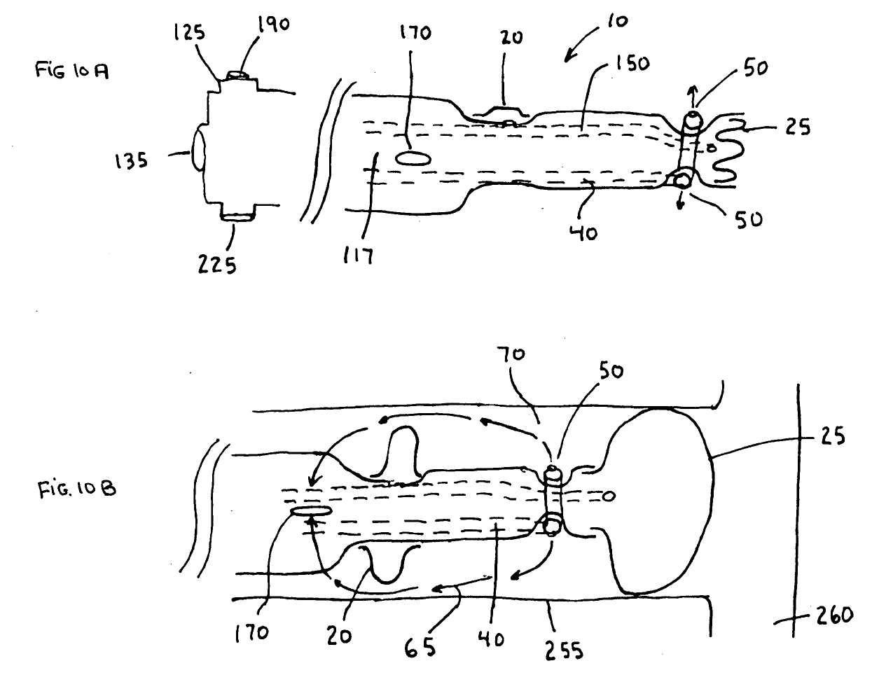

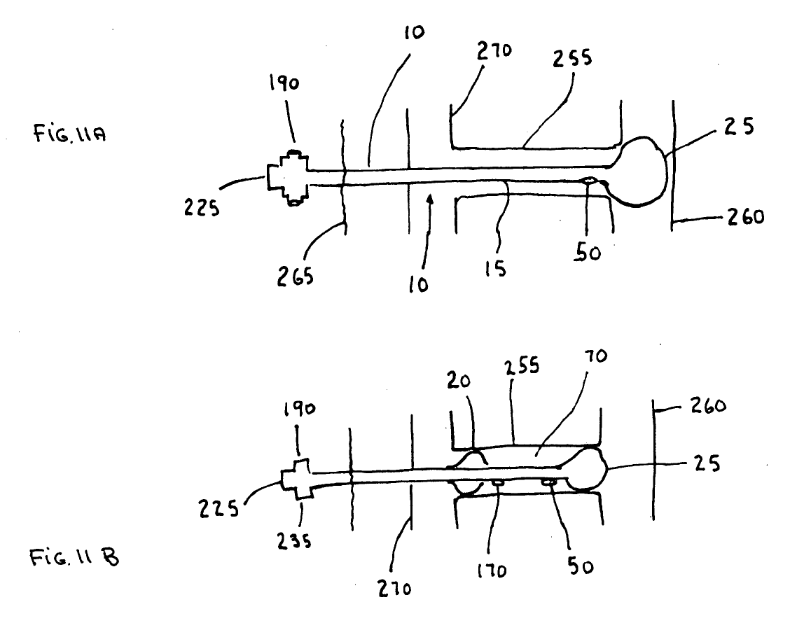

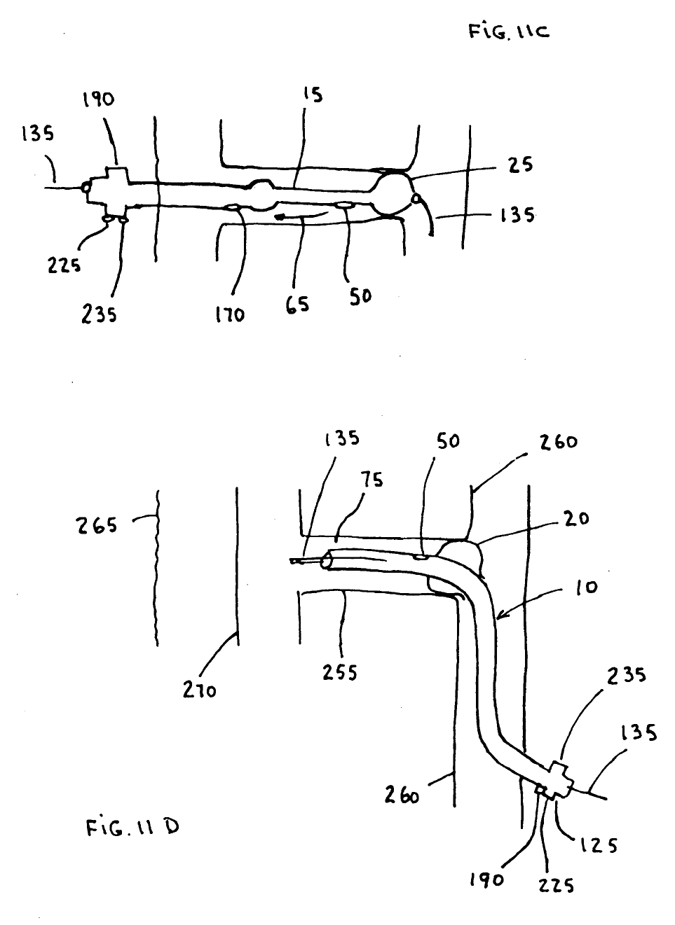

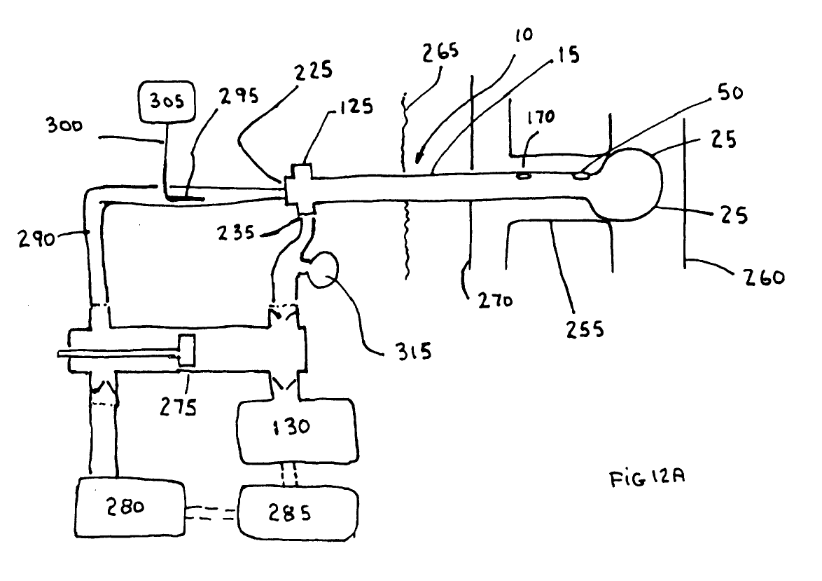

A second preferred embodiment is shown in FIG. 2 where the side jet (55) again is formed from a sclerosant solution that is similar to the first embodiment. In this embodiment, the recirculation pattern (65) directs the flow from the side jet (55) toward the proximal end of the catheter. The proximal balloon (20) can be formed with a diameter that is somewhat smaller than the distal balloon (25). The distal orifice (100) has been replaced by a proximal orifice (160) that directs a proximal jet (165) towards an exit port (170) or effluent opening (170) located proximal to the proximal balloon (20). In a manner similar to that described for the first embodiment, the recirculating sclerosant is drawn toward the proximal jet (165) and the proximal jet (165) helps to drive the effluent (115) out of the shaft lumen (45).

In a third embodiment the sclerosing medium can be a heated saline solution, heated sterile water, heated mixture, or heated sclerosing solution that causes the wall of the vein to become necrotic upon or soon after its contact. The heated saline or fluid can be heated via any means used in the medical device industry for heating a fluid to a temperature greater than 45 degrees Celsius such as with an electrical resistance heater. The heated fluid could be warmed to a preferred temperature that ranges from 45-55 degrees C. for improved ablative effectiveness; temperatures higher than this can also be used with increased effectiveness but with the potential for neurological pain. The distal jet (105) can also have heated saline or also it could be saline at normal body temperature to help return the vein to an equilibrium state that is closer in temperature to a normal body temperature. The heated fluid agent can effectively ablate a large superficial vein and can also make direct contact with the perforator veins.

Just prior to or following treatment of the superficial vein via the delivery catheter (10) of the present invention it may be beneficial to place an occluding member to provide an absolute blockage that will ensure that thrombus will not extend from the treated superficial vein to the deep venous system. There is currently not a suitable occluding member that is easy to place and is small in diameter and flexible. The present implantable occlusion member of one embodiment is a biodegradable occlusion balloon (175) that is inflated with saline. Upon inflation, the balloon creates an instantaneous occlusion such that the balloon diameter exceeds the natural diameter of the vein by 10-100%. After the balloon has effectively made forcible contact with the vessel wall, cellular ingrowth will begin to cause the wall of the balloon to become attached to the venous wall via cellular ingrowth, and the infiltration of cells into the balloon will cause the balloon to become compromised and the saline to be removed naturally. The balloon will form a small flexible member that will be broken down by the body over time.

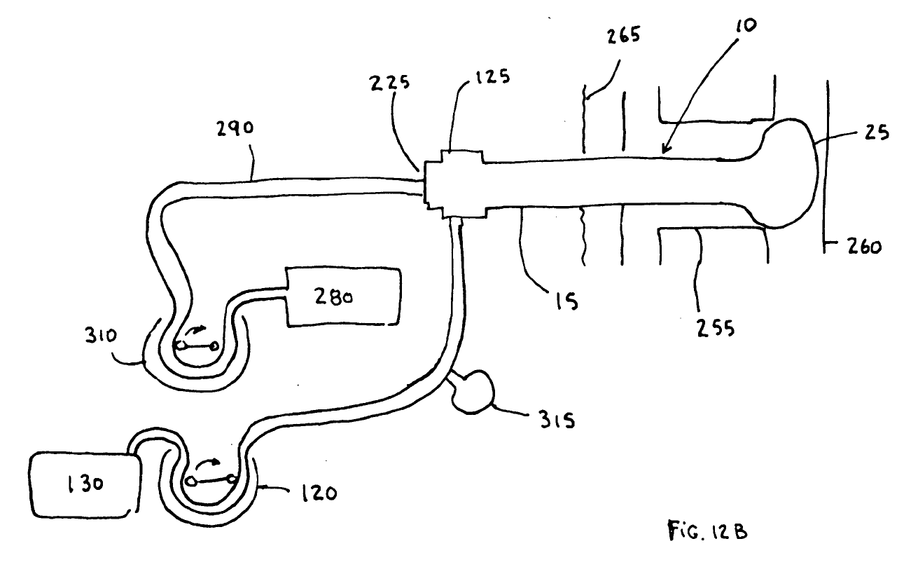

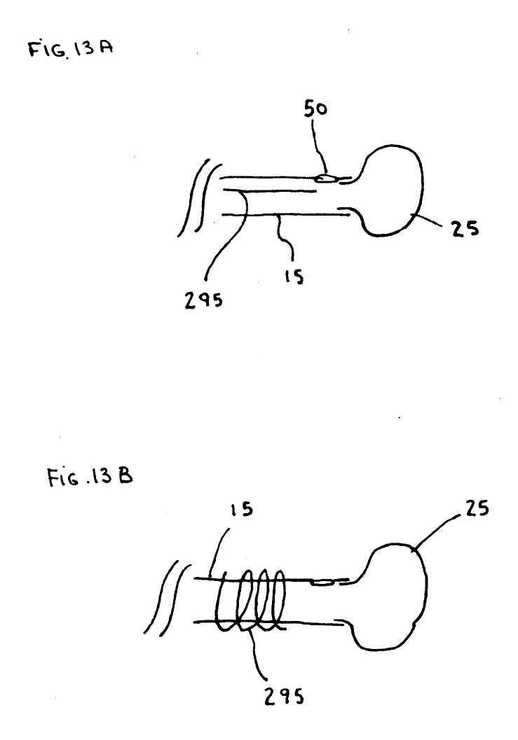

FIG. 3 shows one embodiment of the present biodegradable occlusion balloon (175). It is formed from a material that is easily broken down by the body. Such materials include but are not limited to pericardial tissue, collagen, fibrin, polyethylene glycol, polylactic acid, polyglycolic acid, and others. The implantable occlusion balloon (175) has a proximal valve (180) that is intended to retain the saline once the catheter shaft (15) has been detached. The proximal valve (180) can be duck-bill valve formed from two small flat members of a biodegradable material as mentioned earlier for the balloon material. During delivery the catheter shaft (15) is temporarily attached to the balloon via a reversible release attachment (185) that can be a threaded screw, a releasable clamp, or other mechanical release mechanism. An inflation lumen (150) allows the balloon to be inflated with saline from the inflation port (190). The catheter manifold (125) has a movable element (195) that controls the release of the balloon after it has been inflated. The outer balloon surface (200) can be a textured surface to allow for enhanced tissue ingrowth and form a firm attachment with the vein wall (80).

The occlusion balloon (175) and the catheter shaft (15) can be constructed to allow passage of a guidewire (135) there through as shown in FIG. 4. The balloon has a distal seal (205) that allows passage of the guidewire (135) through the balloon. The distal seal (205) can be formed from a biodegradable material that is formed into a simple duck-bill valve similar to the valve that is located near the proximal end of the balloon. Alternately, a separate biodegradable guidewire tubing can provide for guidewire passage through the balloon as is typical for most angioplasty catheter balloons. The balloon is delivered in a deflated condition as shown in FIG. 5A and is inflated to expand the balloon into the vein lumen (60) extending the vein wall (80) as shown in FIG. 5B. After a few weeks, tissue ingrowth will provide an escape for the contained saline and the balloon will resume a deflated conformation.

To form the implantable occlusion balloon (175), a molding procedure can be utilized using a sheet of biodegradable material that is deformed under increased temperature and pressure and appropriate liquid solvent to form one half of the balloon. Two balloon halves can be attached together using biodegradable glue or sutures to form a complete balloon. The balloon can also be formed via a slurry or solution of collagen or other biodegradable material that is injected into a rotating mold cavity that forces the solution to coat the inside surface of the mold.

The embodiments for a delivery catheter (10) and system described in subsequent figures of the present application including FIGS. 6A to 20F share several reference numerals and also share component descriptions such as those found in the embodiments of FIGS. 1A-2. For the embodiments of the present invention sclerosing fluid can, be a sclerosing liquid including STS and other liquid sclerosants, a heated sclerosing fluid, heated saline, a sclerosing foam, a heated sclerosing foam, steam, alcohol, or other sclerosing fluid. The embodiments of the present invention can have one or more side orifices (50) in the delivery catheter (10) shaft. Several side orifices can distribute sclerosing fluid around the perimeter of the catheter shaft (15). The side orifice (50) in these embodiments can provided a high velocity radial jet of sclerosing fluid that helps to ablate or abrade endothelium and other cell components from the inner lumen of the vessel wall and enhance the effectiveness of the sclerosing fluid. Such a high velocity jet can have a velocity ranging from 30 ft/second to over 300 ft/second. The diameter of a side orifice (50) can range from 0.0015 to approximately 0.040 inches. Lower velocity jets or streams can have a side orifice (50) diameter that is larger than 0.040 inches. Higher velocity jets are associated with side orifice (50) diameters that are on the lower portion of the range. The side orifice (50) can alternately provide a lower velocity jet below 30 ft/second and provide an enhanced recirculation pattern (65) adjacent to the delivery catheter (10) within the lumen of the perforator vein. The side orifice (50) can also provide a very low delivery velocity of only a few cm/minute to ensure that a continual or intermittent new supply of fresh sclerosing fluid is delivered to the treated region of the vein that is being isolated from the deep vein by a distal balloon (25) and also in some embodiments being contained within the treated vein by both a distal and a proximal balloon (20). The side orifice (50) can also have periods when the delivery rate for the sclerosing fluid has been halted or has zero velocity. The pressure supplied by the sclerosant supply pump (310) (see FIG. 14A) can range from a low infusion pressure of 15-30 psi to provide a low flow rate from the side orifice (50). A larger pressure from the supply pump (310) can range from 30 psi to 10,000 psi to create a high velocity jet that will cause vascular trauma to the inside lumen of the perforator vein. The exit port or effluent opening (170) of subsequent embodiments of the present invention including FIGS. 6A to 20F can range in diameter from 0.020 to 0.080 inches or larger. One or more effluent openings (170) can be placed around the perimeter of the catheter shaft (15) to help remove sclerosing fluid in a more evenly spaced or distributed pattern.

The delivery catheter (10) in any embodiment of the present invention can have an ultrasound marker (210) located near the distal end (75) of the catheter as shown in one embodiment of FIG. 6A to help identify the location of the delivery catheter (10) using ultrasound guidance. The ultrasound marker (210) can be a piezoelectric band that is located around the catheter shaft (15) and activated by an electrical signal generated by an ultrasound power supply (215). The ultrasound power supply (215) provides an oscillating electrical signal to a lead wire (220) that extends throughout the catheter shaft (15) from the manifold (125) to the ultrasound marker (210). The electrical signal can also be transmitted from the ultrasound power supply (215) to the ultrasound marker (210) via a wireless RF signal. The natural frequency of the ultrasound marker (210) can be matched to the frequency or multiple of the frequency being used during the placement of the catheter or the therapeutic treatment being performed by the delivery catheter (10). Often diagnostic or therapeutic procedures are performed at frequencies that range from 1 to 40 MHz. The ultrasound marker (210) can also be a passive oscillator such as a coil spring, leaf spring, or other oscillating material that has a natural frequency that is similar or a multiple of the frequency of the ultrasound transducer being used during the interventional procedure. The ultrasound marker (210) can also be a material that reflects ultrasound energy in a particular direction or with a particular reflection pattern that allows the delivery catheter (10) to be easily seen under ultrasound. Such ultrasound markers (210) include foam materials, materials of significant density difference from the surrounding materials, planar materials, or shapes that reflect or absorb ultrasound energy in a manner that is different from the surrounding tissues and materials.

FIGS. 6A-20F show embodiments for a delivery catheter (10) that can be used for direct access through the skin and into the lumen of a perforator vein or other vein of the body to effect its ablation. The catheter has a very low profile of approximately 3 French (1 mm) diameter although it can range in diameter from approximately 2 to 6 French for treatment of perforator veins and can be approximately 2-12 French for other veins of the leg. The embodiment of FIG. 6A has the distal balloon (25) partially folded and inserted into the distal end (75) of the catheter shaft in its small diameter or deflated configuration. The distal balloon (25) can be made of polyurethane, silicone, or other elastomeric material; alternately it could be formed from a nondistendable material such as polyethylene terephthalate (PET), polyethylene, polyvinyl chloride, or other materials commonly used for making balloons for medical catheters. The distal balloon (25) is a sealing balloon and forms a seal with the vein to be ablated along the perimeter of the balloon. Sclerosing fluid enters the catheter manifold (125) at the fluid inlet port (225) and travels down the side tube (40) to the side orifice (50). The side tube (40) can be a metal hypodermic tube or a plastic tube such as a polyimide tube or the side tube (40) can be a separate lumen in a multilumen tubing that forms the catheter shaft (15). The side orifice (50) directs the sclerosing fluid outwardly against the vein wall; more than one side orifices (50) can be provided to direct the sclerosing fluid outwards. The number of side orifices of the delivery catheter (10) embodiments of the present invention can range from one to approximately six. A balloon inflation port (190) on the manifold (125) directs inflation medium such as contrast medium, air, CO2, or other fluid through the inflation lumen (150) to the distal balloon (25). Inflation of the balloon with a gas such as CO2 can help to reduce the profile of the catheter shaft (15) due to low viscosity and ease of transmission of the inflation fluid through a smaller inflation lumen (150). In the embodiment of FIGS. 6A and 6B the catheter shaft (15) only has a distal balloon (25); the balloon is shown in its inflated or large diameter configuration in FIG. 6B. This distal balloon (25) prevents sclerosant from moving distally within the perforator vein or other vein and potentially entering into the deep venous system. Upon delivery of the sclerosant to the vessel wall, the sclerosing fluid is therefore directed proximally along the outside of the catheter shaft (15) and inside of the perforator vein such that it is delivered into the superficial vein.

FIG. 7A shows another embodiment with the balloon deflated or small diameter configuration for delivery to the perforator or other vein and in an inflated or large diameter configuration in FIG. 7B during sclerosant delivery to ablate the vein. In one embodiment the catheter has a guidewire lumen (230) which follows over a guidewire (135) that has been place into the perforator vein or into the vein that is desired for access. Access to the perforator vein could be via direct access through the skin (265) (see FIG. 11A) and into the perforator vein lumen (60). Alternately, access to the perforator vein could be through the deep venous system or through the superficial venous system. In this embodiment, when it is delivered directly through the skin or from a superficial vein, a distal balloon (25) is located on the catheter shaft (15); this distal balloon (25) could be placed into the deep vein or in the perforator vein near to the deep vein to prevent sclerosant fluid from entering into the deep vein. The distal balloon (25) can be bonded onto the outside of the catheter shaft (15) via standard methods and is inflated via an inflation lumen (150) extending through the catheter shaft (15). It is understood that if the catheter is advanced into the perforator vein from the deep vein, that the balloon becomes a proximal balloon (20) as shown in FIG. 7C and would be located one or more centimeters more proximally on the catheter shaft (15) from the distal end (75) to protect the deep vein from exposure to the sclerosing fluid. If necessary an additional balloon could also be placed distal to this proximal balloon (20) to provide an isolated region of vessel to be treated by the sclerosing fluid as shown in other embodiments. The delivery catheter (10) as shown in FIGS. 7A-7C has one or more side orifices (50) that are located on the catheter shaft (15) to direct sclerosing fluid outwardly onto the vein wall (80) and cause ablation. The outwardly shooting jet emanating from the side orifice (50) can cause endothelial denudation and additional trauma to the vein wall (80) and help to enhance the effectiveness of the sclerosing fluid. The sclerosing fluid can also be heated as described in later embodiments. One or more effluent openings or exit ports (170) can be added to this embodiment in a manner similar to that described in other embodiments such as the embodiment of FIGS. 8A and 8B.

FIGS. 8A and 8B show an embodiment that is similar to that shown in FIGS. 6A and 6B except that it includes an effluent opening (170) located in the catheter shaft (15) to allow the sclerosing fluid located in the perforator vein to be removed. The effluent opening (170) joins to an effluent lumen (117) that extends along the catheter shaft (15) and exits at the effluent port (235) located on the manifold (125). More than one effluent opening (170) can be provided along the catheter shaft (15) to provide a more uniform removal of effluent (115) around the perimeter of the catheter shaft (15). The embodiments of the present invention could contain from one to approximately six effluent openings (170). The sclerosant is supplied to the fluid inlet port (225) of the catheter manifold (125) where it travels down the side tube (40) to the side orifice (50) to direct a stream or jet onto the perforator vein wall (80). To inflate the distal balloon (25) inflation fluid or medium (inflation fluid could be a gas) is injected into the balloon inflation port (190); it travels down the inflation lumen (150) to the distal balloon (25) located at the distal end (75) of the catheter. Inflation of the balloon to a large diameter configuration as shown in FIG. 8B within the deep vein or the perforator vein will protect the deep vein from exposure to sclerosing fluid.

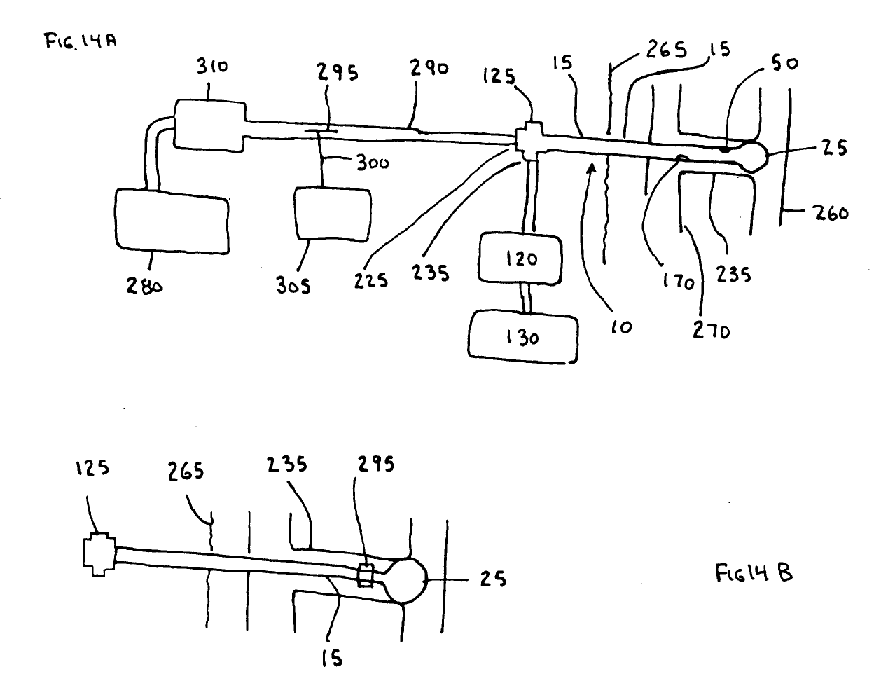

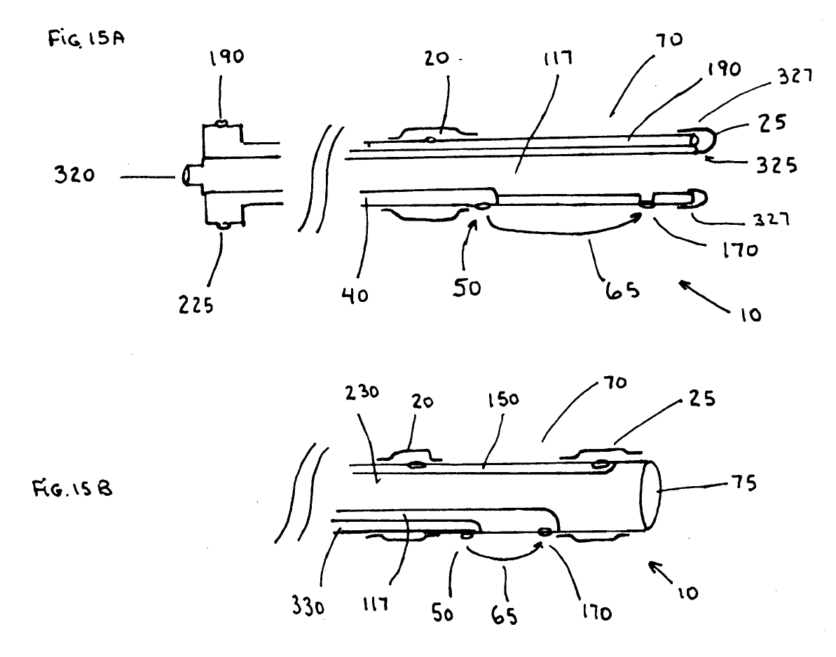

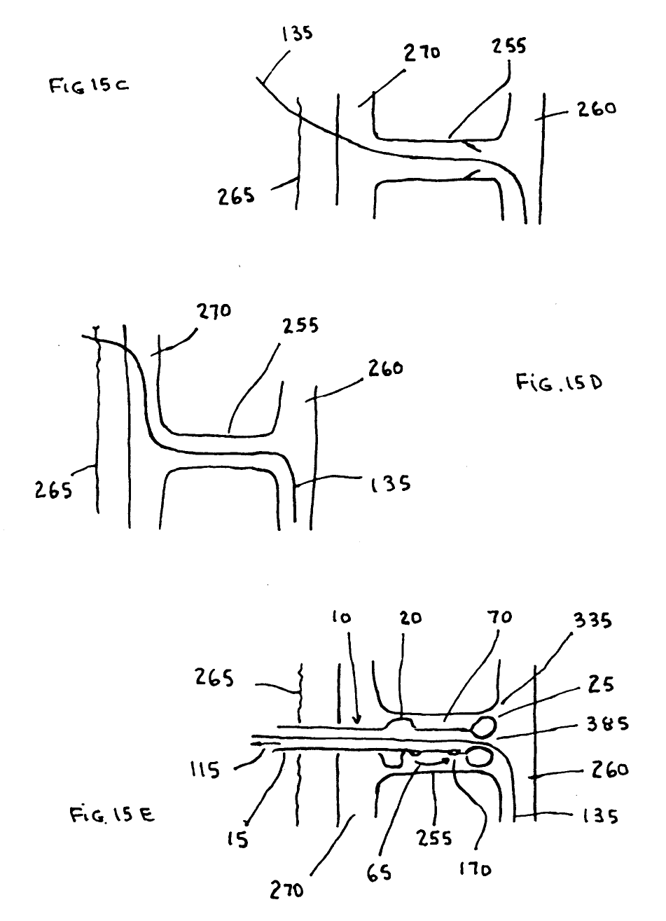

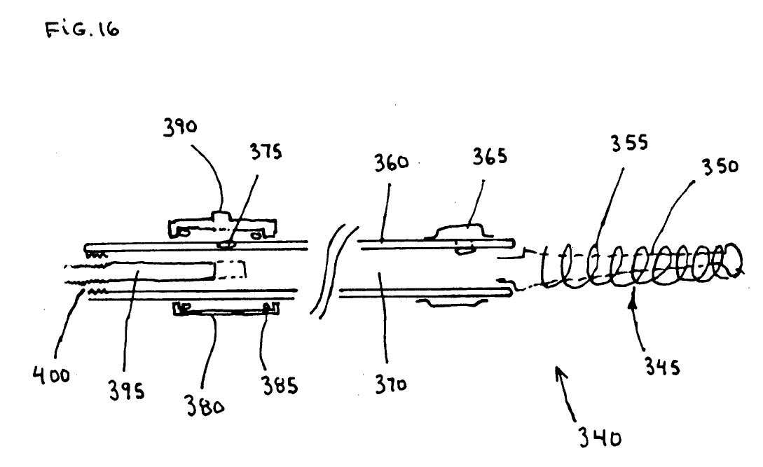

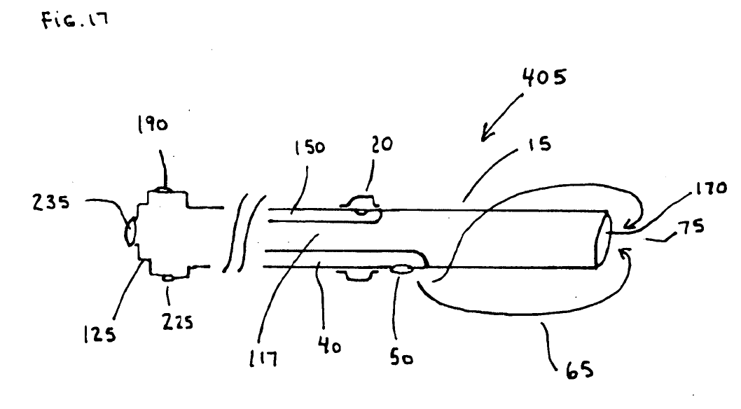

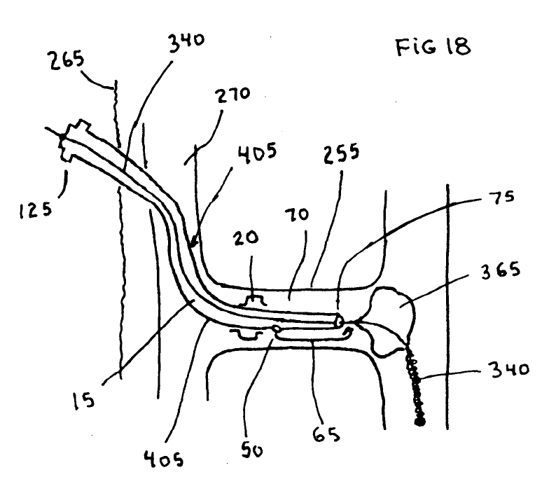

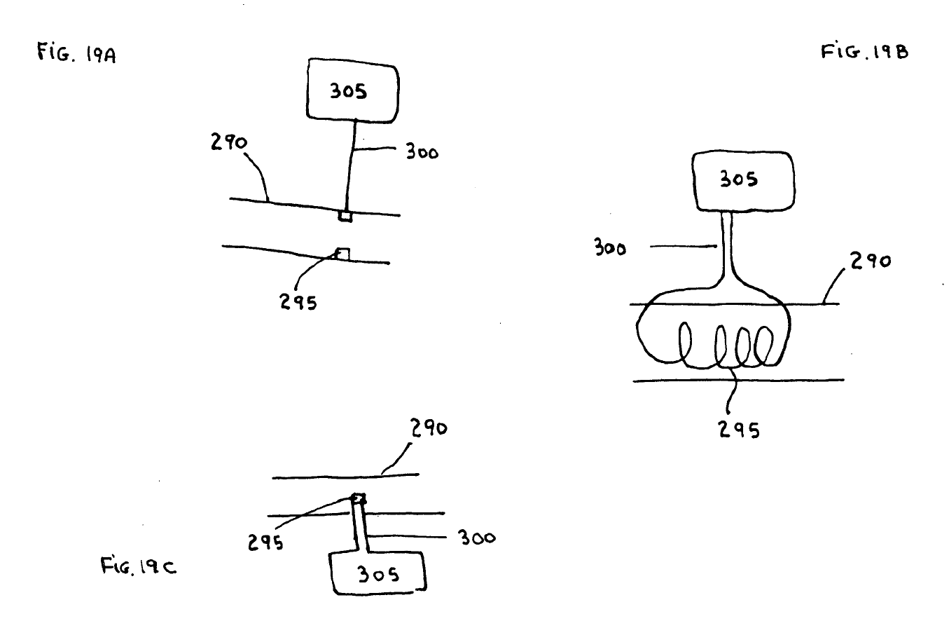

FIGS. 9A and 9B show an embodiment that has both a distal balloon (25) and a proximal balloon (20) and is intended primarily for ablation of a perforator vein but can be used on any vein of the body. The balloons can be inflated individually or together via one or more balloon inflation ports (190). The distal balloon (25) and proximal balloon (20) both serve as sealing balloons forming a seal with the vein around the perimeter of the balloon. Sclerosant enters the fluid inlet port (225) on the manifold (125) and is directed along the catheter shaft (15) to the side orifice (50). In this embodiment the side orifice (50) for delivery of sclerosant is located between the two balloons and can be located adjacent or near to the distal balloon (25). The sclerosant is directed radially via the side orifice (50) against the vein wall (80). The sclerosing fluid has a recirculation pattern (65) between the two balloons and is then removed from the effluent opening (170) located between the two balloons which can be nearer the proximal balloon (20). The radial velocity of the sclerosant along with its recirculation pattern (65) contained between the two balloons helps to enhance the effectiveness of the sclerosing fluid by preventing its dilution. The effluent (115) is then directed out of the effluent port (235) located on the manifold (125). Containment of the sclerosing fluid and providing removal of the effluent (115) sclerosing fluid provides safety to the patient by reducing the risk for trauma to the deep venous system and reducing the risk for deep venous thrombosis forming in the deep venous system. The proximal balloon (20) is separated from the distal balloon (25) by a distance that ranges from 3 mm to approximately 100 mm. For treatment of perforator veins, the distance between balloons is preferably 3 mm to 20 mm due to the generally smaller length for perforator veins. The diameter for perforator veins can range from 1.5 to approximately 10 mm, and the diameter for the balloons for use in such perforators can have similar diameters.

The distal balloon (25) and the proximal balloon (20) can have a roughened coating applied to the outer surface as shown in FIGS. 9C and 9D. The roughened surface (240) can be formed by coating the balloon with a suspension of particles contained in a polymeric solution. The particles can be formed, for example, from ceramic, silica, metal, or small polymeric particles that could be spherical or preferably have a sharp edges. The particles can then be suspended into a solution, for example, of silicone, latex, polyurethane, or other elastomeric polymer that is dissolved in a solvent for the polymer. The balloon itself can also be formed from a thermoplastic elastomer such as polyurethane, or it can be formed from silicone, latex, or other suitable balloon forming material. The balloon can be dipped into the suspension containing the particles along with an elastic polymer; evaporation of the solvent will leave the particles distributed and bonded along the outer surface of the balloon. The particle diameter can range from approximately 0.001 to 0.020 inches in diameter. Either the distal balloon (25), the proximal balloon (20), or both balloons can be formed with a roughened surface (240).

As shown in FIG. 9C the balloon can be inflated within the vein such that the roughened surface (240) comes into contact with the vein wall (80). The balloon can be moved in an axial direction (245) to cause trauma to the vein wall (80). Exposure of the vein wall (80) to such trauma will cause the vein wall (80) to become more susceptible to the sclerosing fluid and cause a greater amount of trauma to the endothelial surface as well as the medial layer and also the adventitial layers of the vein wall (80). The result is a greater tendency for vein wall (80) necrosis and fibrosis and long term durability of venous ablation. Alternately, the balloon shaft can be rotated in a rotational direction (250) thereby causing the roughed balloon to create trauma to the vein wall (80) resulting in an increased susceptibility of the vein wall (80) to necrosis from the sclerosing fluid. Such roughed balloons can be used in any of the delivery catheter (10) embodiments of the present invention. The catheter shaft (15) can be moved in an axial direction (245) or rotational direction (250) via digital movement by the operator at a slow rate of approximately 0.1-5 cm/second. Alternately, the catheter can be moved in a rotational direction (250) or axial direction (245) via a mechanical rotating or axial vibrating means at a higher rate of rotational or axial velocity greater than 5 cm/second.

FIGS. 10A and 10B show yet another embodiment for the delivery catheter (10) for ablating a perforator vein (255) or other vein of the body. In this embodiment the sclerosing fluid is delivered from the fluid inlet port (225) through a side tube (40) to one or more side orifices (50) that direct the sclerosant outward onto the vein wall (80). FIG. 10 shows the delivery catheter (10) in a small diameter or deflated configuration for the balloon. A distal balloon (25) which is inflated into the deep vein (260) or a distal portion of the perforator vein (255) as shown in FIG. 10B prevents sclerosant from contacting the deep vein (260). A proximal balloon (20) is located within the perforator vein (255). The proximal balloon (20) in this embodiment has a smaller diameter than the distal balloon (25). The sclerosing fluid travels around the outside perimeter of the proximal balloon (20) and exits through the effluent opening (170) located proximal to the proximal balloon (20). The recirculation pattern (65) provided by the isolating distal balloon (25) and the smaller diameter proximal balloon (20) ensures that the sclerosant travels into direct contact with the perforator vein wall (80) along the entire perimeter of the vein and thereby ensures direct contact of the sclerosing fluid with the entire vein wall (80) and therein provides a more durable ablation of the vein wall (80). The effluent (115) exits the effluent opening (170), travels down the effluent lumen (117) and out of the effluent port (235).

FIG. 11A shows the method of ablating a perforator vein (255) wherein the delivery catheter (10) enters the perforator vein (255) directly from access across the skin (265). The catheter is advanced distally into the perforator vein (255), preferably into or near the deep vein (260). The distal balloon (25) is inflated and tensioned tightly into the perforator vein (255). Sclerosant is injected into the fluid inlet port (225) and it is delivered via one or more side orifices (50) to the perforator vein (255). The sclerosant passively or convectively flows toward the superficial vein (270) where sclerosing of the superficial vein (270) can be accomplished along with the perforator vein (255). The sclerosing fluid can be heated if desired to enhance its effectiveness.

FIG. 11B shown the method for ablating a perforator vein (255) with sclerosant by using a catheter having two balloons on its distal portion. The catheter enters through the skin (265) and the distal balloon (25) is inflated via the balloon inflation port (190) within or near the deep vein (260). The proximal balloon (20) is inflated within the perforator vein (255). The proximal balloon (20) is similar in diameter to the distal balloon (25). Sclerosant enters through the fluid inlet port (225) and is delivered out of the one or more side orifices (50) which are located near the distal balloon (25) and between the two balloons. The sclerosant is contained between the two balloons. The sclerosant is removed from between the two balloons via one or more effluent openings (170) which are located just distal to the proximal balloon (20) and between the two balloons. The rate of effluent (115) flow of sclerosing fluid is maintained in balance with the rate of inflow from the side orifice (50). The balance of flow is controlled by the control pump (120) as shown in FIG. 1B. The rate of outflow can be balanced such that it is equal to, greater than, or less than the inflow of sclerosant fluid from the side orifices (50). Following the ablation procedure, the sclerosant contained in the recirculation region (70) between the two balloons can be removed and replaced with a saline liquid or other biocompatible liquid.

FIG. 11C shows an embodiment of the delivery catheter (10) for ablation of a perforator vein (255) that is similar to that of 11B except that the one or more effluent openings (170) are located proximal to the proximal balloon (20). The proximal balloon (20) is smaller in diameter than the distal balloon (25). During use, the sclerosant exits the one or more side orifices (50) and travels proximally across the outside of the proximal balloon (20). This movement and recirculation pattern (65) places the sclerosant into contact with the entire perimeter of the vein wall (80). The radially directed jet shooting out of the side orifice (50) assists in removing endothelium and other cellular tissue from the vein wall (80) resulting in vessel wall trauma and enhances the effectiveness of the sclerosant ablation.

FIG. 11D shows a method of delivery for the catheter from the deep vein (260) and extending into the perforator vein (255). The catheter follows over a guidewire (135) that has been place into the deep vein (260) and advanced into the perforator vein (255); placement can be made via percutaneous access to the vein. A proximal balloon (20) located on the catheter shaft (15) approximately 0.5-4 cm from the distal end (75) of the catheter is inflated with inflation medium to isolate the deep vein (260) and prevent sclerosant fluid from coming into contact with the deep vein (260). Sclerosing fluid is directed radially out of the side orifice (50) and onto the wall of the perforator vein (255). The sclerosing fluid can travel toward the superficial vein (270) for removal. Alternately, a second balloon placed at or near the distal end (75) of the catheter similar to the device embodiment shown in FIG. 11B can contain the sclerosant fluid. An effluent opening (170) can be located in the catheter shaft (15) to remove the sclerosant fluid and prevent its delivery to the superficial vein (270) if desired. Alternately, the effluent (115) can be removed from the distal end (75) of the catheter

FIGS. 12A and 12B show embodiments of a supply/control pump (275) used with any of the embodiments of the present invention described in this patent specification or otherwise contemplated. FIG. 12A shows one embodiment for the supply/control pump (275) that can be used along with the delivery catheter (10) of the present invention. This pump is a piston pump and can deliver a pulsatile flow of sclerosant to the fluid inlet port (225) of the catheter. A single piston pump can be used to both provide sclerosant fluid from the supply reservoir (280) to the delivery catheter (10) as well as remove the effluent (115) fluid from the delivery catheter (10) to the collection reservoir (130). Alternately, individual pumps can be used separately for the supply pump (310) to supply sclerosing fluid from the supply reservoir (280) to the delivery catheter (10) and a control pump (120) such as shown in FIG. 1A to control the flow of effluent (115) fluid removal from the delivery catheter (10) to the collection reservoir (130) as described in FIG. 1A. The effluent (115) fluid can be returned to the delivery catheter (10); the collection reservoir (130) and the supply reservoir (280) can form a common collection/supply reservoir (285) (280). The piston pump takes sclerosant fluid from the supply reservoir (280) and delivers it to a supply tube (290). The supply tube (290) connects to the fluid inlet port (225) located on the manifold (125) of the delivery catheter (10). A heating member (295) can be located within the supply tube (290) to receive energy via a transmission conduit (300) from an energy supply (305) that can generate heat in order to heat the fluid such as the sclerosant fluid prior to delivery or during delivery to the vein lumen (60). Other components of the system use reference numerals as described in previous embodiments.

FIG. 12B shows an embodiment wherein a roller pump is being used as a supply pump (310) to provide the supply of sclerosant from the supply reservoir (280) and delivery to the fluid inlet port (225) of the delivery catheter (10). Similarly a roller pump can be used as a control pump (120) to control the rate of sclerosant effluent (115) from the delivery catheter (10) and delivery to the collection reservoir (130). The control pump (120) can take sclerosing fluid or other fluid from the effluent port (235) and deliver it to a collection reservoir (130). A pressure transducer (315) can be placed in the effluent tube to ensure that the effluent (115) flow has not been blocked or that effluent (115) flow is equal to the sclerosant inlet flow. A drop in pressure as sensed by the pressure transducer (315) is indicative that a blockage of the effluent opening (170) may have occurred. The sclerosing fluid effluent (115) can be returned back to the supply tube (290) if desired as described in FIG. 1B. As an alternate, other pumps such as a centrifugal pump can also be used with the present invention.

FIGS. 12A, 13A and 13B show a heating member (295) such as a resistance heating wire or coil that is electrically connected via an electrical wire or transmission conduit (300) to an energy supply (305) such as a battery or an energy supply (305) that can obtain its energy from a standard wall outlet. The heating member (295) can be placed within the supply tube (290) as shown in FIG. 12A to affect a temperature increase of the sclerosant fluid. Alternately, the heating member (295) can be placed within the catheter shaft (15) near the distal end (75) of the delivery catheter (10) as shown in FIG. 13A. The heating member (295) or resistance element can alternately be placed on the outside of the catheter shaft (15) such that it is in direct contact with the fluid within the perforator vein (255) as shown in FIG. 13B; thus sclerosing fluid that is injected into the perforator vein (255) via the side orifice (50) will be heated to a temperature that is higher than the temperature of the incoming sclerosing fluid and preferably higher than the standard body temperature.

In one embodiment the temperature increase required to provide an improved effectiveness for the sclerosant is 5-25 degrees Celsius above normal body temperature of 37 degrees Celsius, and preferably 5-25 degrees above normal body temperature, and more preferably 10-25 degrees above body temperature. At this temperature the patient does not experience pain or neuropathy during treatment in the lower leg such as found during venous ablation using laser or RF therapy in lower leg vessels. The temperature of the sclerosant should be at least 10 degrees above body temperature of 37 degrees Celsius in order to generate trauma to the vessel wall associated with the increase in temperature. In another embodiment the temperature can be raised higher than this, and can be more effective, but the patient may experience pain and neuropathy which can require the use of tumescent anesthesia. In this embodiment the temperature of the sclerosant fluid is raised 20-40 degrees Celsius above the normal body temperature; the effectiveness of the sclerosant fluid is enhanced and the patient does not experience pain or neuropathy in many venous ablation applications including the ablation of the greater saphenous vein located in the thigh.

The temperature of the sclerosant fluid can be raised to over 70-80 degrees Celsius if desired and remain within the teachings of the present invention; it is recognized that the present invention is not limited by the upper limit of sclerosing fluid temperature. The sclerosant fluid can be raise to a temperature of 80-100 degrees to accomplish an enhanced effectiveness for the sclerosant fluid. The sclerosant fluid can be converted to steam at temperatures of approximately 100 degrees Celsius and higher without deviating from the present invention. The sclerosant can be heated water or heated saline which can be converted to steam via heat transfer or electromagnetic coupling from a heating member. Upon condensation of the steam, removal of the liquid sclerosant can be accomplished using the means described in the present invention. The presence of the distal and proximal balloons maintains the heated zone such that it is localized and does not cause injury to neighboring veins or tissues including protection of the deep venous system. One of the advantages of using sclerosant fluid and not heating it above approximately 47 to 60 degrees C. is the elimination of tumescent anesthesia as required by standard RFA and EVLT. Any of the embodiments found in this invention can be used with any of the sclerosants discussed and at any temperature. The balloons found in any of the embodiment of the present invention can be used to control the delivery of sclerosant fluid and localize the zone of ablation such that the deep veins or other segments of veins or neighboring tissues are protected from the sclerosant ablation.

The amount of energy needed to raise the temperature of the sclerosing fluid from room temperature or 22 to 47 degrees C., for a contained volume of 2 milliliters is approximately 50 calories. A typical disposable battery can provide an energy level of 0.14-0.36 MJ/kg. A disposable battery of approximately % lb weight can provide over 3000 calories of energy and can be used to provide the power requirements for heating the sclerosing fluid. The amount of energy required of a battery to heat the sclerosant fluid to temperatures higher than 50 degrees Celsius can also be provided from a disposable battery or from an external power source. The amount of energy required is proportional to the change in temperature of the sclerosant fluid from its initial room temperature and is also proportional to the amount of sclerosant fluid required to compete the venous ablation treatment.

Heating the sclerosant fluid to temperatures ranging from 47 degrees Celsius to 100 degrees Celsius or higher is anticipated in the present invention. The heated sclerosant is more effective in producing a venous ablation than room temperature or body temperature sclerosant. The increased effectiveness of heated sclerosant allows the concentration of sclerosant to be reduced while still accomplishing an adequate ablation not only of the intimal layer of the vein wall but also including a traumatic effect onto the medial and adventitial layers of the vein wall. Typically sclerosants such as STS and POL are sold in concentrations of 1% and 3% and are used at the higher concentrations in veins with diameters ranging from 5 mm diameter and higher. Lower concentrations are often used in spider veins and smaller diameter superficial veins. With the present invention, the effectiveness of the 1% concentration of heated sclerosant in veins of larger diameters above 5 mm is equal or greater than the effectiveness of 3% concentrations of sclerosant at standard room temperature. This improved effectiveness of the heated sclerosant provides two benefits. First, from a safety standpoint, the lower concentration of sclerosant reduces the concern for any embolization of the low concentration sclerosant into the deep veins or to regions of veins which are functional and are not in need of a sclerosant. The lower concentration of sclerosant fluid is more easily diluted by the blood and reduced in concentration to a level that does not cause vessel trauma. Secondly, the cost of the sclerosant is proportional to its concentration; the lower concentration of sclerosant thereby allows for a reduction in cost for the sclerosant.

The embodiments of the present invention have been described with specific features including the number of balloons which can range from one to three, the number of opening for sclerosant effluent (115) removal, number of side orifices, the location of the effluent openings (170) with respect to various balloons, and the configuration of the balloons. For example it is within the scope of the present invention to have more than two balloons located near the distal end (75) of the delivery catheter (10) in order to generate two or more recirculation regions (70) with their respective recirculation patterns (65). One region, for example, could have a sclerosing fluid contained between two balloons and another region could have, for example, a saline solution between another two balloons that is intended to provide a post ablation cleaning of the sclerosant out of the vessel, or a pre-ablation preparatory treatment with a fluid of choice including a sclerosant fluid. Several types of sclerosants or sclerosant fluids have been described that are suitable to use with the embodiments of the present invention including liquid and foam detergents, heated saline, heated sclerosant, steam, alcohol sclerosant, hypertonic solutions, and other sclerosant fluids. The temperature of sclerosant or sclerosant fluid can also be adjusted within the scope of the present invention between very cold or cryogenic temperatures below zero degrees centigrade (to freeze or provide trauma to the vessel wall) to temperatures above the boiling point of water (above 100 degrees centigrade) such as used for the generation of steam. It is understood that the invention is not limited to those specific embodiments described herein but that any of the features of one embodiment can be used with another embodiment without deviating from the present invention.

FIGS. 14A and 14B show the sclerosant delivery catheter (10) and sclerosant delivery system being used for direct ablation of a perforator vein (255) or other vein via access through the skin (265) directly into the lumen of the vein. The delivery catheter (10) can cross the skin (265) and enter the lumen of the perforator anywhere along the length of the perforator vein (255). A distal balloon (25) is located at the distal end (75) of the catheter to isolate the deep vein (260) and ensue that sclerosant cannot enter or contact the wall of the deep vein (260). A side orifice (50) directs sclerosant radially outward into contact with the vein wall (80). A side opening provides a removal of the sclerosant that can be balanced in flow rate to the rate of sclerosant inflow. The sclerosant effluent (115) exits the effluent port (235) on the manifold (125) and has a flow rate that is controlled via a control pump (120) which pumps the fluid into a collection reservoir (130).

To improve the effectiveness of the sclerosant, it is anticipated that one preferred embodiment has a heating member (295) to increase the temperature of the inlet sclerosant to a temperature higher than normal body temperature (37 degrees C.) or the temperature found in the supply reservoir (280) that contains the sclerosing fluid for delivery. Increase of the sclerosant temperature by 5-25 degrees C. above normal body temperature will improve the effectiveness for ablating the vein wall (80) and will not cause unwanted pain or neuropathy associated with current thermal ablation modalities such as RFA and EVLT. The solubility of many sclerosant molecules such as STS and polidocanol in water or saline is decreased at higher temperatures thereby causing the sclerosant molecule to be more active in its sclerosing effect. Also, the higher temperature sclerosant will penetrate further into the wall of the vein to provide enhanced sclerosing effect.

The supply pump (310) delivers the sclerosant via a supply tube (290) from the supply reservoir (280) to the fluid inlet port (225) of the delivery catheter (10). In one embodiment a heating member (295) contained within the supply tube (290) or near the delivery tube distal end (75) is used to elevate the sclerosant temperature prior to delivery to the fluid inlet port (225). The heating member (295) can include any heating element capable of heating the sclerosing fluid or fluid contained within the vein being treated.

An additional embodiment for the present delivery catheter (10) is shown in FIG. 15A and 15C-E. This embodiment can travel over a guidewire (135) that is advanced from the superficial vein (270) at a location that is distant from the perforator vein (255) to be treated as shown in FIG. 15D. The guidewire (135) (such as a 0.012-0.014 inch diameter guidewire) is then advanced through the perforator vein (255) and into the deep vein (260). Alternately, the guidewire (135) can enter directly into the perforator vein (255) from the skin (265) adjacent to the perforator vein (255) as shown in FIG. 15C. The delivery catheter (10) is then advanced over the guidewire (135) and into the perforator vein (255) as shown in FIG. 15E with the guidewire (135) extending through the guidewire port (320). During advancement of the catheter, the deflated annular shaped distal balloon (25) on the distal end (75) of the catheter can provide a passage for the guidewire (135) there through and the guidewire (135) can remain in place if desired during the sclerosing treatment of the perforator vein (255). The distal balloon (25) is able to form a seal with the guidewire (135) upon inflation such that the guidewire lumen (230) can be used for guidewire passage as well as removal of sclerosant effluent (115) as the effluent (115) travels through the effluent opening (170) and out of the effluent lumen (117) as shown in FIGS. 15A and 15E. The distal balloon (25) has a diameter that ranges from 2-7 mm and is formed from an elastomeric material such as polyurethane or silicone. Inflation of the balloon will generate both a distal guidewire seal (385) between the balloon and the guidewire (135) as well as a perforator seal (335) between the balloon and the perforator vein wall (80). The effluent lumen (117) is thereby shared between the sclerosant and the guidewire (135). Alternately, the guidewire (135) can be removed once the catheter has reached its location in the perforator vein (255). With the guidewire (135) removed the annular-shaped distal balloon (25) can form a seal upon itself via the balloon inner surface (325) of the annular distal balloon (25) as well as the balloon outer sealing surface (327) with the distal perforator vein wall (80) or the deep vein wall (80); the entire guidewire lumen (230) can then be used as an effluent lumen (117) for removal of the effluent (115) sclerosant. Sclerosant fluid is delivered via the fluid inlet lumen (330) and out of the side orifice (50); sclerosant fluid then moves toward and out of the effluent opening (170). As described in previous embodiments, a proximal balloon (20) can be positioned approximately 1-3 cm proximal to the distal balloon (25) to contain the sclerosant that is exiting the side orifice (50) and forming a recirculation pattern (65) between the two balloons for ablating perforator veins (255). The distance between balloons can be larger for treating other veins such as other superficial veins (270). If it is not necessary in some patients to protect the superficial vein (270) from sclerosant fluid, the proximal balloon (20) would not be required and would not be an element of one embodiment of the present invention but would be an element of another embodiment of the present invention. The catheter can also be advanced over the guidewire (135) from a remote site with initial entry into the deep venous system and advancing the catheter over the guidewire (135) into the perforator vein (255) from the deep vein (260). A larger balloon diameter and large balloon spacing can be used with the catheter of this embodiment for applications for treating veins of larger diameter including superficial veins (270).

An alternate embodiment for an OTW catheter that has a small profile and two balloon located approximately 1-3 cm is shown in FIG. 15B for a similar application for treating a perforator vein (255) as described in FIGS. 15A and 15C. The distal balloon (25) is intended to form a seal with the distal end (75) of the perforator vein (255) near the junction of the perforator vein (255) with the deep vein (260). The proximal balloon (20) is inflated to contain the sclerosant between the balloons in a recirculation region (70) with a recirculation pattern (65) and to prevent sclerosant migration into the superficial veins (270) if required. If a larger superficial vein (270) is to be treated, the balloon spacing can be enlarged along with the balloon diameter.

Another embodiment of the present invention is the distal occlusive guidewire (340) shown in FIG. 16. This occlusive guidewire (340) is intended to provide the initial entry into the superficial vein (270) at a remote site or entry into the perforator vein (255) via direct access into the skin (265) adjacent the perforator vein (255). The occlusive guidewire (340) can be used with other over the guidewire (OTW) catheter embodiments of the present patent specification or other OTW catheters used for other diagnostic or therapeutic purposes. The occlusive guidewire (340) has a distal flexible region (345) having a flexible inner component (350) and an outer coil (355) that is attached to the guidewire body (360) and allows the occlusive guidewire (340) to be formed in to a curved shape that can be directed through the venous vasculature in a manner similar to other clinically available guidewires. Other guidewire flexible region (345) construction including polymeric coated guidewires with a metal core can be used without deviating from the present invention. The occlusive guidewire (340) body is hollow and can be formed from stainless steel, Nitinol, or other metals used in guidewire construction. A distal guidewire balloon (365) is located near the distal end (75) of the guidewire and is in fluid communication with the guidewire inflation lumen (370). The guidewire inflation lumen (370) extends proximally through the guidewire body (360) and is in fluid communication with a guidewire inflation opening (375). A removable manifold (380) is placed over the guidewire inflation opening (375) and forms a guidewire seal (385) with the guidewire body (360). Inflation of the distal guidewire balloon (365) is obtained through the guidewire inflation port (390) located on the removable manifold (380). Upon inflation of the distal guidewire balloon (365), an occlusion element (395) is advanced within the hollow guidewire body (360) and across the guidewire inflation opening (375) and held in place via a locking means (400) (such as a threaded connection) to form a fluid tight seal of the occlusion element (395) across the guidewire inflation opening (375). Removal of the removable manifold (380) can then be performed after the balloon is inflated while maintaining the distal balloon (25) in an inflated state. A catheter can then be advanced over the occlusive guidewire (340) body to a position near or adjacent to the guidewire balloon (365). Balloon deflation is attained by releasing the locking means (400), for example, by unscrewing the threaded locking means (400) to allow removal of an inflation fluid from the guidewire opening. The removable manifold (380) can be placed over the guidewire body (360) to allow a vacuum to be applied to the guidewire inflation lumen (370) via a syringe inserted into the guidewire inflation port (390) on the removable manifold (380). The inflation fluid can be a liquid contrast medium solution, saline solution, a gas, or a mixture of various inflation fluid media.

The distal occlusive guidewire (340) can be used with another embodiment of the present invention, a proximal occlusive delivery catheter (405), shown in FIG. 17 to form a system. This proximal occlusive delivery catheter (405) is a very low profile and very flexible catheter shaft (15) that is able follow over a guidewire through the very flexible and distortable venous vasculature. The catheter shaft (15) near the open distal end (75) has an inner diameter that is approximately 0.002 inch larger than the diameter of the approximately 0.014 inch diameter guidewire. A larger diameter guidewire can be used for larger vessels found in the peripheral vasculature. The shaft can be made from typical flexible plastic materials used in forming small coronary angioplasty catheters such as pebax, polyethylene, and others. The outer diameter of the distal end (75) of the catheter shaft (15) can be made to be approximately 0.020 inches (range 0.016-0.045 inches). A proximal balloon (20) (“proximal” is used here to differentiate the proximal balloon (20) of the occlusive delivery catheter (405) from the guidewire distal balloon (25) of the distal occlusive guidewire (340) of the embodiment of FIG. 16) is located a small distance of 1-3 cm (range 0.5-10 cm) from the distal end (75) of the catheter for treating perforator or small diameter veins. The distance of the proximal balloon (20) from the distal end (75) of the catheter can be larger for treating larger diameter and larger length veins. A balloon inflation lumen communicates the proximal balloon (20) with the balloon inflation port (190) located on the catheter manifold (125). Sclerosant fluid is delivered through the fluid inlet port (225) on the manifold (125), travels along the catheter shaft (15) within a side tube (40), and exits through the side orifice (50) located just distal to the proximal balloon (20). Sclerosant is delivered to the recirculation region (70) with a recirculation pattern (65) from the side orifice (50) to the open distal end (75) of the catheter. The open distal end (75) of the catheter serves as an effluent opening (170) to allow entry of sclerosant into the effluent lumen (117). The sclerosant effluent (115) travels out of the catheter in the effluent lumen (117) which also can serve as a guidewire lumen. The sclerosant exits the effluent port (320) which also can serve as a guidewire port.

One method of use of the distal occlusive guidewire (340) of the embodiment of FIG. 16 and the proximal occlusive delivery catheter (405) of the embodiment of FIG. 17 is shown in FIG. 18. The occlusive guidewire (340) is advanced into the venous vasculature using a Seldinger technique at a location that is either remote or adjacent to the perforator vein (255) to be treated as shown in FIG. 18. The wire is advanced into the perforator vein (255) and further advanced until the distal guidewire balloon (365) is positioned in or near the deep vein (260). The distal guidewire balloon (365) is then inflated and the locking means (400) is activated to hold the balloon in an inflated position and the guidewire is retracted to pull the distal balloon (25) into occlusive contact with the junction of the perforator vein (255) with the deep vein (260) or within the perforator vein (255). The removable manifold (380) is removed from the guidewire. Verification of position is obtained using ultrasound guidance; an ultrasound marker (210) as described earlier can be placed on the guidewire if necessary. Alternately, to assist in advancing the guidewire from the superficial vein (270) to the perforator vein (255), a steerable catheter, currently available on the market, can be placed initially into the superficial vein (270) wherein a small “J” curve is placed into the steerable catheter, in situ. The distal occlusive guidewire (340) of the present invention is then passed through this steerable catheter and into the perforator vein (255) and advanced further into the deep vein (260). The steerable catheter is then removed prior to advancing the proximal occlusive deliver catheter over the occlusive guidewire (340).

Continuing the method as shown in FIG. 18, the proximal occlusive delivery catheter (405) is advanced over the occlusive guidewire (340) until the catheter distal end (75) is positioned near the guidewire balloon (365). The proximal balloon (20) of the occlusive delivery catheter (405) is inflated within the perforator vein (255) to form a recirculation region (70) between the catheter proximal balloon (20) and the distal guidewire balloon (365) where sclerosant fluid will form a recirculation pattern (65). Sclerosant fluid is delivered via the fluid inlet port (225) of the catheter manifold (125) and exits the side orifice (50) just distal to the catheter proximal balloon (20). The sclerosant fluid follows a recirculation pattern (65) between the two balloons and exits the catheter distal end (75). Sclerosant fluid does not enter the deep venous system due to occlusive protection provided by the distal guidewire balloon (365). Sclerosant fluid does not flow into the superficial vein (270) due to occlusive protection provided by the catheter proximal balloon (20). The proximal balloon (20) of the catheter can be omitted from the present invention if it is not required to prohibit the sclerosant fluid from entry into the superficial vein (270). This occlusive delivery catheter (405) and occlusive guidewire (340) can also be used via entry into the perforator vein (255) from the deep venous system. Also, this system can be used to ablate large or small veins in the superficial system; the balloon diameter would be adjusted to be approximately 5-30% larger than the vein diameter in order to make a good seal with the vein. The spacing between the proximal and distal balloon (25) is determined by the length of vein that is being treated, the diameter of the vein, whether it is effective to pull or retract the catheter during treatment, and the time required for exposure of the sclerosant to the vein wall (80) to accomplish an effective vein ablation. For example, a 3% STS solution could require approximately 0.5-5 minutes of exposure to effectively ablate a vein (less time for smaller vein diameter). For heated sclerosant, the time required for effective vein ablation is reduced by over 30%.

Heating the sclerosant improves the effectiveness for causing vein ablation. Various methods for heating the sclerosant have been contemplated. In one embodiment the heating member (295) can be a bipolar electrode that is comprised of one or more pairs of electrodes that are located in the supply tube (290) as shown in FIG. 19A. Alternately, a single unipolar electrode can form the heating member (295) and a second electrode can be placed on the outside of the skin or other location on or within the patient limb that is being treated. An RF energy supply (305) provides an oscillating voltage that is in direct contact with the sclerosing fluid within the supply tube (290). Polar molecules such as water molecules are caused to heat under the exposure to such RF energy. Alternately, a coil as shown in FIG. 19B can be placed within or around the supply tube (290) can serve as the heating member (295) and RF electromagnetic energy is generated in the coil via an RF energy supply (305). This energy is then coupled to the water contained within the supply tube (290) to cause temperature increase. Another method as shown in FIG. 19C for rapidly heating the sclerosing fluid is to place it into contact with a laser probe which serves as a heating member (295) that receives its energy, for example, from a diode laser energy supply (305) that provides electromagnetic energy of a wavelength of approximately 1319, 1320 or 1470 nm. These wavelengths will rapidly be absorbed by water molecules and will result in an increase in temperature.

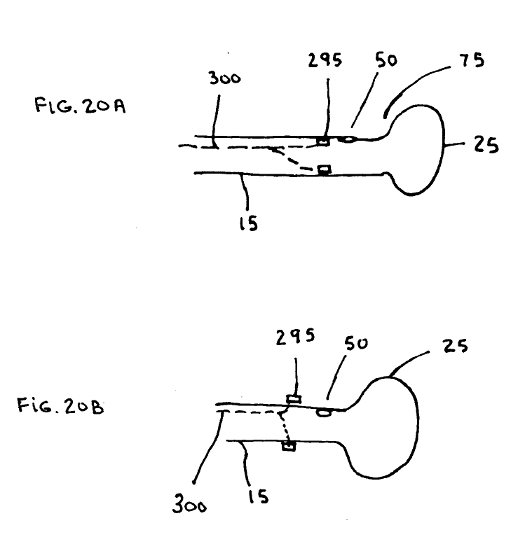

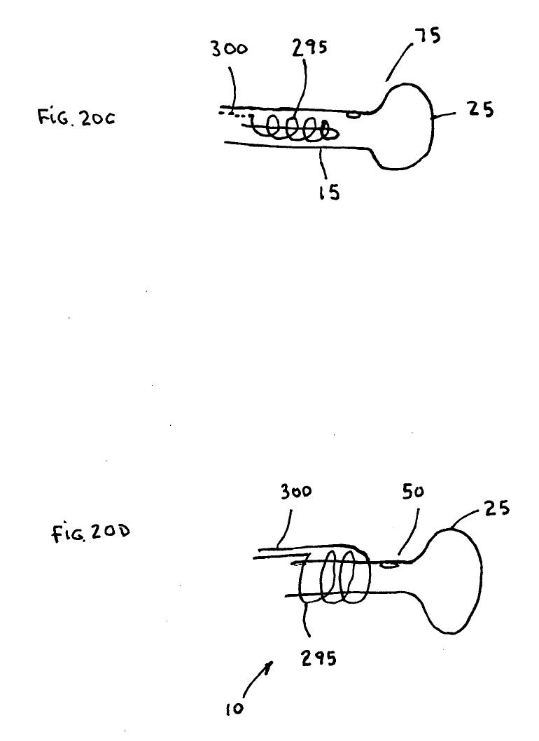

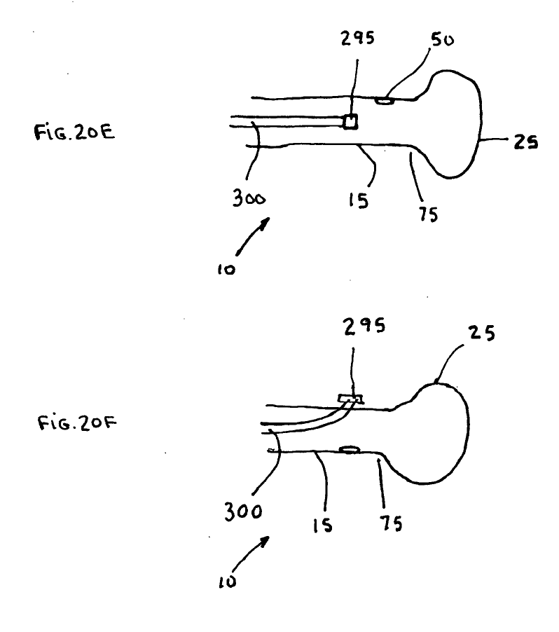

In another embodiment the heating member (295) such as an RF electrode or Laser probe can be placed near the distal end (75) of the delivery catheter (10) as shown in FIGS. 20A-20F. In FIG. 20A a bipolar RF electrode heating member (295) is placed within the catheter shaft (15) near the side orifice (50). FIG. 20B shows the bipolar electrode heating member (295) located on the outside surface of the catheter shaft (15) in direct contact with the fluid that would be found outside the catheter shaft (15) but within the perforator vein to be treated. FIG. 20C shows a coil RF electrode heating member (295) placed within the catheter shaft (15) near the distal end (75). The coil couples via electromagnetic induction to the polar molecules such a water contained within the sclerosing fluid to cause heating. The RF coil can equally well be placed on the outside of the delivery catheter (10) shaft as shown in FIG. 20D. A laser probe can be placed within the catheter shaft (15) as shown in FIG. 20E. The probe on the distal end (75) of the catheter receives light energy from the laser energy supply (305) through a fiber optic energy transmission conduit (300) that extends throughout the catheter shaft (15). The laser light energy can be approximately 1319, 1320 or 1470 nm in wavelength to absorb within water and heat the water located within the sclerosing fluid. Alternately, the laser probe heating member (295) can be located outside the catheter shaft (15) into the fluid contained within the perforator vessel as shown in FIG. 20F. A wavelength of approximately 810, 940, or 980 nm can be used absorb readily in blood components such as hemoglobin and cause them to heat resulting in trauma to the wall of the perforator vein to be treated.

The laser probe, RF electrode, or electrical heating members (295) can be used to generate steam which then functions as the sclerosant or sclerosing fluid. Water can be delivered to the distal end (75) of any of the delivery catheters (10) of the present invention presented in this patent application where it is exposed to a heating member (295) which converts the water into steam. The steam can range in temperature from approximately 100 degrees C. to several degrees higher or lower. The steam is then delivered to the vessel wall where it condenses within the vessel lumen delivering its latent heat of vaporization and cause that vessel wall to become adequately traumatized leading to vessel wall necrosis. The steam is released from the side orifice (50) and is prevented from delivery into the deep venous system or distal to the catheter via the distal balloon (25). A proximal balloon (20) can provide a recirculation pattern (65) for the steam in the recirculation region (70) and prevent the steam from traveling in a proximal direction or into a vein that is not intended to be ablated by the steam. The balloons can be formed from silicone, polyurethane, thermoplastic elastomer, or other polymer that can withstand temperatures above 100 degrees centigrade without degradation. Alternately, the heating member (295) can be place outside of the catheter shaft and convert water located in the blood that surrounds the catheter to become vaporized.

It is understood that any of the embodiments described in FIGS. 1A-20F can include or comprise any other element from other embodiments described in this specification or shown in any of the drawings. For example, the sclerosant fluid used in an embodiment can be liquid or foam detergent, hot saline, steam, alcohol, heated sclerosant, hypertonic solution, or any other sclerosant or sclerosing fluid that is deliverable via a catheter. The catheter of the present invention can have one balloon, two balloons, or three or more balloons, or no balloons. Other embodiments are contemplated and the present invention is not limited to only those embodiments that are described or drawn.

1. A catheter for delivery of a sclerosant fluid to a vein lumen within the body for causing ablation of the vein, said catheter comprising;
A. an elongated catheter shaft having a sclerosant fluid lumen that provides passage for the sclerosant fluid therethrough, said catheter shaft having at least one orifice near its distal end for delivery of the sclerosant to the vein lumen,
B. one or more balloons located near the distal end of said catheter shaft, at least one of said balloons being a sealing balloon, said sealing balloon making contact with the vein along a balloon perimeter,
C. a heating member in fluid communication with said sclerosant fluid lumen, said heating member transferring thermal energy to the sclerosant fluid to raise its temperature at least ten degrees Celsius above normal body temperature of 37 degrees Celsius.

2. The catheter of claim 1 further comprising a second balloon located near the distal end of said catheter shaft, said orifice being located between said sealing balloon and said second balloon.

3. The catheter of claim 1 further comprising at least one effluent opening located near the distal end of said catheter shaft, said effluent opening providing a passage for removal of the sclerosant fluid from the vein into said catheter shaft.

4. The catheter of claim 2 further comprising at least one effluent opening located near the distal end of said catheter shaft, said effluent opening being located between said sealing balloon and said second balloon.

5. The catheter of claim 1 further comprising;
A. an open distal end, said open distal end providing for passage of a guidewire through said catheter shaft, said open distal end also providing an effluent opening for passage of effluent sclerosant fluid from the vein into said catheter shaft,
B. wherein said sealing balloon is an annular shaped sealing balloon located at the distal end of said catheter shaft and residing substantially within said catheter shaft in a noninflated configuration, said annular shaped balloon having an outer sealing surface to seal against the vein and an inner sealing surface to seal against the guidewire.

6. The catheter of claim 1 wherein;
A. said sealing balloon is located proximal to said orifice,
B. said catheter having an open distal end, said open distal end providing an effluent opening for passage of effluent sclerosant fluid from the vein into said sclerosant fluid lumen of said catheter shaft, said open distal end also providing passage for a guidewire throughout said catheter shaft through said sclerosant fluid lumen,
C. said catheter providing passage within said effluent lumen and out of said open distal end of said catheter shaft, said distal occlusive guidewire having a guidewire balloon located near its distal end that is in fluid communication with a guidewire inflation opening, said guidewire balloon having a sealing surface that seals along a guidewire balloon perimeter with the vein.

7. The catheter of claim 1 wherein said orifice directs a sclerosant fluid jet toward said sclerosant fluid lumen in a proximal direction away from said distal end of said catheter shaft, said sclerosant fluid jet generating a stagnation pressure to drive said sclerosant fluid in the proximal direction within said catheter shaft.

8. The catheter of claim 2 wherein said second balloon is smaller than said sealing balloon, said second balloon being smaller than the vein diameter to provide an annular space between its surface and a wall of the vein during inflation.

9. The catheter of claim 1 wherein said heating member is located within said catheter shaft.

10. The catheter of claim 1 wherein said heating member is attached to the outside of said catheter shaft.

11. The catheter of claim 1 wherein said heating member is taken from a group that includes an electrical resistance element, an RF heating electrode, and a Laser probe.

12. The catheter of claim 1 wherein said heating member heats said sclerosant fluid by 10 to 20 degrees Celsius above body temperature.

13. The catheter of claim 1 wherein said heating member heats said sclerosant fluid from approximately room temperature to a temperature ranging from 50 to 100 degrees Celsius.

14. The catheter of claim 1 wherein said heating member heats water within said sclerosant fluid to form steam which exits the orifice into the vein.

15. The catheter of claim 1 wherein said sclerosant fluid is taken from a group that includes, sodium tetradecyl sulfate, sodium morrhuate, heated water, heated saline, heated sclerosant, steam, ethanol, alcohol, hypertonic saline, polidocanol, foam sclerosants, foam sclerosant formed with CO2, foam sclerosant formed with air, microfoam, and heated foam.

16. A catheter for delivery of a sclerosant fluid to a vein lumen within the body for causing ablation of the vein, said catheter comprising;
A. an elongated catheter shaft having a sclerosant fluid lumen that provides passage for the sclerosant fluid therethrough, said catheter shaft having at least one orifice near its distal end for delivery of the sclerosant to the vein lumen,
B. one or more balloons located near the distal end of said catheter shaft, at least one of said balloons being a sealing balloon, said sealing balloon making contact with the vein along a balloon perimeter,
C. at least one effluent opening located near the distal end of said catheter shaft, said effluent opening providing a passage for removal of the sclerosant fluid from the vein into said catheter shaft.

17. A catheter for delivery of a fluid medium to a tubular member of the body for providing a controlled therapeutic or diagnostic treatment of the tubular member, said catheter comprising;
A. an elongated catheter shaft having a fluid medium lumen that provides passage for the fluid medium therethrough, said catheter shaft having at least one orifice near its distal end for delivery of the fluid medium to the tubular member,
B. one or more balloons located near the distal end of said catheter shaft, at least one of said balloons being a sealing balloon, said sealing balloon making contact with the tubular member along a balloon perimeter,
C. a heating member in fluid communication with said fluid medium lumen, said heating member transferring thermal energy to the fluid medium to raise its temperature at least ten degrees Celsius above normal body temperature of 37 degrees Celsius.

18. The method of use for ablating a vein comprising the steps;
A. entering a vein with a catheter having a catheter shaft with at least one balloon located near a distal end of said catheter shaft in a deflated condition,
B. advancing the catheter into the lumen of a vein,
C. inflating at least one balloon located near a distal end of said catheter shaft,
D. activating a heating member located in fluid communication with a sclerosant fluid lumen located within said catheter shaft to heat said sclerosant fluid,
E. activating flow of a sclerosant solution to at least one orifice located near a distal end of said catheter shaft to deliver said sclerosant solution into the lumen of the vein at a temperature that is at least 10 degrees Celsius above body temperature.

19. The method of claim 16 wherein said catheter has two balloons located near each other near the distal end of said catheter shaft, said orifice being located between said balloons.
